(19) United States
(12) Patent Application Publication
(10) Pub. No.: US 2011/0123561 A1
(43) Pub. Date: May 26, 2011

(54) METHODS AND COMPOSITIONS FOR INDUCTION OR PROMOTION OF IMMUNE TOLERANCE

(75) Inventors: Franck BARRAT, San Francisco, CA (US); Robert L. COFFMAN, Portola Valley, CA (US)

(73) Assignee: Dynavax Technologies Corporation, Berkeley, CA (US)

(21) Appl. No.: 12/855,671

(22) Filed: Aug. 12, 2010

Related U.S. Application Data
(63) Continuation of application No. 11/303,588, filed on Dec. 16, 2005, now abandoned.
(60) Provisional application No. 60/637,359, filed on Dec. 17, 2004.

(51) Int. Cl. A61K 39/385; C07H 21/04; A61K 31/7088; A61P 37/02

(52) U.S. Cl. 424/193.1; 536/23.1; 536/24.5; 514/44 R

(57) ABSTRACT

The invention provides non-immunostimulatory polynucleotide antigen conjugates and methods for treating unwanted immune reactions in individuals using the non-immunostimulatory polynucleotide antigen conjugates.

### CROSS REFERENCE TO RELATED APPLICATIONS

This application claims the benefit of U.S. Provisional Patent Application Ser. No. 60/637,359, filed Dec. 17, 2004, which is hereby incorporated by reference herein in its entirety.

### TECHNICAL FIELD

The present invention relates to non-immunostimulatory polynucleotide-antigen conjugates. It also relates to the administration of the non-immunostimulatory polynucleotide-antigen conjugates for treating unwanted immune reactions in individuals.

### BACKGROUND OF THE INVENTION

The immune system provides highly specific and often very protective responses against potentially pathogenic microorganisms. In some cases, however, inappropriate and/or unwanted immune activation can cause injurious processes leading to damage or destruction of one's own tissues. Tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self antigens, or self-tolerance. Complete tolerance is characterized by the lack of a detectable immune response to an antigenic challenge. Partial tolerance is typified by the quantitative reduction of an immune response. Although generally steady state and lifelong, tolerance to particular antigens can be disrupted and result in inappropriate immune activation.

Inappropriate and unwanted immune activation occurs, for example, in autoimmune diseases where antibodies and/or T lymphocytes react with self antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens.

In some cases, powerful immunosuppressive drugs are used to prevent or reduce inappropriate or unwanted immune responses in order to treat patients with an autoimmune disease or with an allogeneic transplant. The infusion of individuals with drugs that prevent or suppress a T-cell immune response does inhibit the unwanted immune activation, but can also result in general immune suppression, toxicity and even death due to opportunistic infections.

One of the primary goals in developing effective therapies against diseases caused by unwanted or tissue damaging immunological reactions such as allograft rejection, autoimmune diseases, and tissue destructive allergic reactions to infectious microorganisms or to environmental antigens, is to specifically suppress or decrease to an acceptable level the intensity of deleterious immune processes without affecting the remainder of the immune system.

There remains a need to identify strategies to control inappropriate and unwanted immune activation. There is a need for the prevention and/or reduction of inappropriate immune activation and response in, for example, autoimmune disease and allergies. There is also a need for the prevention and/or reduction of an unwanted immune response by a host to a transplant and by a donor tissue against a recipient tissue, known as graft-versus-host disease.

All patents, patent applications, and publications cited herein are hereby incorporated by reference in their entirety.

### DISCLOSURE OF THE INVENTION

The invention relates to non-immunostimulatory polynucleotide-antigen conjugates (non-immunostimulatory conjugate or NISC) and methods for regulating unwanted or inappropriate immune responses in subjects using these conjugates, particularly in humans.

In one aspect, the invention provides non-immunostimulatory conjugate (NISC) comprising a non-immunostimulatory polynucleotide linked to an antigen. In certain embodiments, the invention includes compositions which comprise any of the NISCs described herein. The compositions may also include, for example, a pharmaceutically acceptable excipient or any of a number of other components.

In another aspect, the invention provides methods for inducing or promoting peripheral tolerance to an antigen comprising administering to a subject an effective amount of a non-immunostimulatory conjugate. Administration of the NISC in accordance with the invention induces or promotes peripheral tolerance to the antigen in the NISC.

In another aspect, the invention provides methods for ameliorating a symptom of an unwanted immune activation comprising administering to a subject in need thereof an effective amount of a non-immunostimulatory conjugate. Administration of the NISC in accordance with the invention ameliorates a symptom of the unwanted immune activation directed to the antigen in the NISC. In some examples, the unwanted immune activation is an autoimmune response, an allergy, asthma, a graft-versus-host reaction or a graft rejection reaction.

In another aspect, the invention provides methods for suppressing an autoimmune response comprising administering to a subject in need thereof an effective amount of a non-immunostimulatory conjugate. Administration of the NISC in accordance with the invention suppresses an autoimmune response directed to the antigen in the NISC.

In another aspect, the invention provides methods for suppressing a symptom of an autoimmune disease comprising administering to a subject in need thereof an effective amount of a non-immunostimulatory conjugate. Administration of the NISC in accordance with the invention suppresses a symptom of an autoimmune disease involving an immune response to the antigen in the NISC.

In another aspect, the invention provides methods for preventing an autoimmune disease comprising administering to a subject at risk of developing an autoimmune disease a non-immunostimulatory conjugate. Administration of the NISC in accordance with the invention prevents a symptom of an autoimmune disease involving an immune response to the antigen in the NISC.

In another aspect, the invention provides methods for suppressing an allergic response comprising administering to a subject in need thereof an effective amount of a non-immunostimulatory conjugate. Administration of the NISC in accordance with the invention suppresses an allergic response directed to the antigen in the NISC.

In another aspect, the invention provides methods for suppressing an allergy symptom comprising administering to a subject in need thereof an effective amount of a non-immunostimulatory conjugate. Administration of the NISC in accordance with the invention suppresses a symptom of an allergy involving an immune response to the antigen in the NISC.

In another aspect, the invention provides methods for preventing an allergic response comprising administering to a subject at risk of developing an allergic response a non-immunostimulatory conjugate. Administration of the NISC in accordance with the invention prevents an allergic response involving an immune response to the antigen in the NISC.

In some embodiments, the NISC of the invention comprises an autoantigen, an alloantigen or an allergen. In some embodiments, the non-immunostimulatory polynucleotide of the NISC of the invention comprises an immunoregulatory sequence (IRS). In some examples, the IRS is a TLR9 class IRS, a TLR7/8 class IRS or a TLR7/8/9 IRS. In some embodiments, the non-immunostimulatory polynucleotide of the NISC of the invention comprises an antisense molecule or an aptamer.

### BRIEF DESCRIPTION OF THE DRAWINGS

FIGS. 1A-1C depicts graphs showing antigen uptake by murine dendritic cells after incubation with indicated compositions: mixture of OVA and immunostimulatory oligonucleotide 1018 (left) FIG. 1A, immunostimulatory oligonucleotide (1018)-OVA conjugate (center) FIG. 1B, non-immunostimulatory oligonucleotide (1040)-OVA conjugate (right) FIG. 1C.

FIGS. 2A-2H depicts graphs showing expression of maturation markers (CD40 and CD86) on murine dendritic cells after incubation in medium alone (FIGS. 2A-2B) or with the indicated conjugates: OVA-C661 (non-immunostimulatory oligonucleotide) (FIGS. 2C-2D), OVA-1040 (non-immunostimulatory oligonucleotide) (FIGS. 2E-2F), and OVA-1018 (immunostimulatory oligonucleotide) (FIGS. 2G-2H).

### MODES OF CARRYING OUT THE INVENTION

According to the present invention, coupling a non-immunostimulatory polynucleotide with an antigen enhances and/or facilitates uptake of the antigen by antigen presenting cells (APCs) and/or dendritic cells (DCs) with little or no APC or DC activation or maturation. Also, coupling a non-immunostimulatory polynucleotide with an antigen increases antigen presentation by APCs and/or DCs with little or no APC or DC activation or maturation.

Thus, the present invention provides methods in which non-immunostimulatory polynucleotide antigen conjugates (NISCs) are used to regulate unwanted or inappropriate immune responses in individuals, particularly humans. The compositions of the invention comprise a non-immunostimulatory polynucleotide coupled to an antigen, where the antigen is involved in the unwanted immune response. The NISCs of the invention particularly suppress and/or inhibit an unwanted immune response to an antigen. The NISCs of the invention are also of use in inducing or promoting peripheral self-tolerance.

Accordingly, the invention provides methods and compositions for suppressing and/or inhibiting an unwanted immune response to an antigen, including, but not limited to, an autoimmune response, an alloimmune response, an allergic response, and similarly aberrant immune responses, for example, celiac disease. The invention also provides methods for generation of antigen-specific T regulatory cells and methods for inhibiting Th1 and/or Th2 cell differentiation.

The invention also provides methods and compositions for ameliorating symptoms associated with unwanted immune activation, including, but not limited to, symptoms associated with autoimmunity, symptoms associated with alloimmunity, symptoms associated with allergy, and symptoms associated with similarly aberrant immune responses, such as celiac disease. Accordingly, the invention also provides methods for aiding in transplantation, such as reducing graft rejection and/or graft-versus-host (GVH) disease.

The invention also provides methods and compositions to induce or promote peripheral self-tolerance.

Further provided are kits comprising the NISCs of the invention. The kits may further comprise instructions for administering a NISC of the invention for immunoregulation in a subject.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques(Greg T. Hermanson, ed., Academic Press, 1996); and Methods of Immunological Analysis (R. Masseyeff, W. H. Albert, and N. A. Staines, eds., Weinheim: VCH Verlags gesellschaft mbH, 1993).

### DEFINITIONS

The term “immunostimulatory” or “stimulating an immune response” as used herein includes stimulation of cell types that participate in immune reactions and enhancement of an immune response to a specific antigenic substance. An immune response that is stimulated by an immunostimulatory nucleic acid is generally a “Th1-type” immune response, as opposed to a “Th2-type” immune response. Th1-type immune responses are normally characterized by “delayed-type hypersensitivity” reactions to an antigen and activated macrophage function and can be detected at the biochemical level by increased levels of Th1-associated cytokines such as IFN-γ, IL-2, IL-12, and TNF-β. Th2-type immune responses are generally associated with high levels of antibody production, especially IgE antibody production and enhanced eosinophils numbers and activation, as well as expression of Th2-associated cytokines such as IL-4, IL-5 and IL-13.

The term “immunostimulatory nucleic acid” or “immunostimulatory polynucleotide” as used herein refers to a nucleic acid molecule (e.g., polynucleotide) that effects and/or contributes to a measurable immune response as measured in vitro, in vivo and/or ex vivo. Examples of measurable immune responses include, but are not limited to, antigen-specific antibody production, secretion of cytokines, activation or expansion of lymphocyte populations such as NK cells, CD4+T lymphocytes, CD8+T lymphocytes, B lymphocytes, and the like. Immunostimulatory nucleic acid sequences are known to stimulate innate immune responses, in particular, those response occur through TLR-9 signalling in the cell. Generally, an immunostimulatory nucleic acid sequence includes at least one CG dinucleotide, with the C of this dinucleotide being unmethylated.

The term “conjugate” refers to a complex in which a non-immunostimulatory polynucleotide and an antigen are coupled. Such conjugate couplings include covalent and/or non-covalent linkages.

The term “non-immunostimulatory polynucleotide” as used herein refers to a nucleic acid molecule (e.g., polynucleotide) that does not effect or contribute to a measurable immune response as measured in vitro, in vivo and/or ex vivo. In particular, a non-immunostimulatory polynucleotide stimulates little, if any, APC or DC activation or maturation. Indicators, and assays for indicators, of APC and DC activation and maturation are known in the art.

The term “immunoregulatory sequence” or “IRS” as used herein refers to a nucleic acid sequence that inhibits and/or suppresses a measurable innate immune response as measured in vitro, in vivo and/or ex vivo. The term “immunoregulatory polynucleotide” or “IRP” as used herein refers to a polynucleotide comprising at least one IRS that inhibits and/or suppresses a measurable innate immune response as measured in vitro, in vivo and/or ex vivo. Inhibition of a TLR, e.g., TLR-7, 8, or 9, includes without limitation inhibition at the receptor site, e.g., by blocking ligand-receptor binding, and inhibition of the downstream signal pathway after ligand-receptor binding. Examples of measurable innate immune responses include, but are not limited to, secretion of cytokines, activation or expansion of lymphocyte populations such as NK cells, CD4+T lymphocytes, CD8+T lymphocytes, B lymphocytes, maturation of cell populations such as plasmacytoid dendritic cells and the like.

The term “immunoregulatory compound” or “IRC”, as used herein, refers to a molecule which has immunoregulatory activity and which comprises a nucleic acid moiety comprising an IRS. The IRC may consist of a nucleic acid moiety that comprises more than one IRS, consists of an IRS, or has no immunostimulatory activity on its own. The IRC may consist of a polynucleotide (a “polynucleotide IRC”) or it may comprise additional moieties. Accordingly, the term IRC includes compounds which incorporate one or more nucleic acid moieties, at least one of which comprises an IRC, covalently linked to a non-nucleotide spacer moiety.

As used interchangeably herein, the terms “nucleic acid,” “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, deoxythymidine, and deoxycytidine. A nucleotide is a phosphate ester of a nucleoside.

The term “3′” generally refers to a region or position in a polynucleotide or oligonucleotide 3′ (downstream) from another region or position in the same polynucleotide or oligonucleotide. The term “3′ end” refers to the 3′ terminus of the polynucleotide.

The term “5′” generally refers to a region or position in a polynucleotide or oligonucleotide 5′ (upstream) from another region or position in the same polynucleotide or oligonucleotide. The term “5′ end” refers to the 5′ terminus of the polynucleotide.

The term “peptide” generally refers to polypeptides that are of sufficient length and composition to effect a biological response, e.g., antibody production or cytokine activity whether or not the peptide is a hapten. Typically, the peptides are at least six amino acid residues in length. The term “peptide” further includes modified amino acids (whether or not naturally or non-naturally occurring), such modifications including, but not limited to, phosphorylation, glycosylation, pegylation, lipidization and methylation.

A “delivery molecule” or “delivery vehicle” is a chemical moiety which facilitates, permits, and/or enhances delivery of a NISC to a particular site and/or with respect to particular timing.

An “individual” or “subject” is a vertebrate, such as avian, and is preferably a mammal, more preferably a human. Mammals include, but are not limited to, humans, primates, farm animals, sport animals, rodents and pets.

An “effective amount” or a “sufficient amount” of a substance is that amount sufficient to effect beneficial or desired results, including clinical results, and, as such, an “effective amount” depends upon the context in which it is being applied. An effective amount can be administered in one or more administrations.

“Suppression” or “inhibition” of a response or parameter includes decreasing that response or parameter when compared to otherwise same conditions except for a condition or parameter of interest, or alternatively, as compared to another condition.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder. Especially in the autoimmune disease context, as is well understood by those skilled in the art, palliation may occur upon regulation or reduction of the unwanted immune response. Further, palliation does not necessarily occur by administration of one dose, but often occurs upon administration of a series of doses. Thus, an amount sufficient to palliate a response or disorder may be administered in one or more administrations.

As used herein, the term “comprising” and its cognates are used in their inclusive sense; that is, equivalent to the term “including” and its corresponding cognates.

As used herein, the singular form “a”, “an”, and “the” includes plural references unless indicated otherwise. For example, “an” antigen includes one or more antigens.

Compositions of the Invention

The invention provides polynucleotide-antigen conjugates wherein the polynucleotide facilitates or enhances uptake of the antigen by dendritic cells (DCs) and/or antigen presenting cells (APCs) with little or no DC or APC activation or maturation. Alternatively, the invention provides polynucleotide-antigen conjugates wherein the polynucleotide increases antigen presentation by DCs and/or APCs with little or no DC or APC activation or maturation. The polynucleotides in such conjugates are non-immunostimulatory polynucleotides. Accordingly, such conjugates are referred to herein as “non-immunostimulatory conjugates” (NISCs). Upon administration, conjugates of the invention can lead to tolerance to the administered antigen since the conjugates do not promote APC and/or DC cells activatation or maturation.

The conjugates contain polynucleotides which stimulate little or no DC or APC maturation. The non-immunostimulatory polynucleotides include, but are not limited to, (a) a polynucleotide containing an immunoregulatory sequence (IRS), (b) a polynucleotide with a particular activity (e.g., an aptamer or an antisense polynucleotide) but which are non-immunostimulatory, and (c) polynucleotides without a particular known activity and which are non-immunostimulatory, i.e., oligonucleotides that are neither (a) or (b).

Compositions of the invention comprise an NISC alone (or a combination of two or more NISCs). Compositions of the invention may also comprise an NISC in conjunction with another agent, such as a second unconjugated antigen, suppressive cytokine (e.g., IL-10, TGF-beta) or other immunosuppressive agents. Compositions of the invention may comprise an NISC and a pharmaceutically acceptable excipient. Pharmaceutically acceptable excipients, including buffers, are described herein and well known in the art. Remington: The Science and Practice of Pharmacy, 20th edition, Mack Publishing (2000).

Non-Immunostimulatory Polynucleotides

As described herein, non-immunostimulatory polynucleotides of the NISCs facilitate or enhance uptake of the antigen by cells, particularly dendritic cells (DCs) and/or antigen presenting cells (APCs). These polynucleotides facilitate or enhance antigen uptake but stimulate little or no cell activation or maturation. This facilitated or enhanced antigen uptake results in increased antigen presentation by the APC and/or DC. In some instances, non-immunostimulatory polynucleotides facilitate or enhance antigen presentation without the polynucleotides stimulating cell activation or triggering cell maturation.

In accordance with the present invention, a non-stimulatory polynucleotide of the NISC may be an immunoregulatory polynucleotide (IRP) or an immunoregulatory complex (IRC) which contain at least one immunoregulatory sequence (IRS) as described in copending U.S. Application Ser. No. 60/606,833 and U.S. application Ser. No. 11/212,297 (hereby incorporated by reference in their entirety). In some instances, an IRS comprises a 5′-G,C-3′ sequence. In some instances, an IRS includes at least one TGC trinucleotide sequence at or near the 5′ end of the polynucleotide (i.e., 5′-TGC). In some instances, an IRS comprises a 5′-GGGG-3′ sequence. In some instances, an IRS does not comprise a 5′-GGGG-3′ sequence. Accordingly, in some instances, an IRP or IRC does not comprise a 5′-GGGG-3′ sequence. In some instances, an IRP or IRC comprising a 5′-GGGG-3′ sequence is particularly effective when used in the single-stranded form. In some instances, an IRP or IRC comprising a 5′-GGGG-3′ sequence is particularly effective when made with a phosphorothioate backbone.

As demonstrated in copending U.S. application Ser. No. 11/212,297 and U.S. Application No. 60/606,833, particular IRPs and IRCs inhibit TLR-7 and/or TLR-8 dependent cell responses. Also, particular IRPs and IRCs inhibit TLR-9 dependent cell responses, and particular IRPs and IRCs inhibit TLR-7/8 dependent cell responses and TLR-9 dependent cell responses. As used herein, “TLR-⅞” refers to “TLR-7 and/or TLR-8.” Accordingly, as used herein, “TLR-7/8/9” refers to “(TLR-7 and/or TLR-8) and TLR-9.” Certain IRPs do not inhibit TLR4 dependent cell responses.

Non-immunostimulatory polynucleotides are defined by the absence of stimulatory activity of innate immune responses. They have been described in the art and the absence of stimulatory activity may be readily measured using standard assays which indicate various aspects of an innate immune response, such as cytokine secretion, antibody production, NK cell activation, B cell proliferation, T cell proliferation, and dendritic cell maturation. DC maturation can be evaluated by the up-regulation of various markers at the cell surface including, but not limited to, costimulatory molecules such as CD40, CD80 and CD86.

Immunostimulatory nucleic acids and other stimulators of an innate immune response have been described in the art and their activity may be readily measured using standard assays which indicate various aspects of an innate immune response, such as cytokine secretion, antibody production, NK cell activation, B cell proliferation, T cell proliferation, dendritic cell maturation. See, e.g. Krieg et al. (1995) Nature 374:546-549; Yamamoto et al. (1992) J. Immunol. 148:4072-4076; Klinman et al. (1997) J. Immunol. 158:3635-3639; Pisetsky (1996) J. Immunol. 156:421-423; Roman et al. (1997) Nature Med. 3:849-854; WO 98/16247; WO 98/55495; WO 00/61151 and U.S. Pat. No. 6,225,292. Accordingly, these and other methods can be used to identify, test and/or confirm sequences, polynucleotides and/or compounds which lack effective immunostimulatory activity, for example those which do not activate APC or DC and those which do not stimulate APC to mature. For example, the effect of NISCs can be determined when cells or individuals in which an autoreactive immune response has been stimulated.

As is clearly conveyed herein, it is understood that, with respect to formulae described herein, any and all parameters are independently selected. For example, if x=0-2, y may be independently selected regardless of the values of x (or any other selectable parameter in a formula). Preferably, the IRP or IRC which comprises the IRS is a polynucleotide with at least one phosphorothioate backbone linkage.

An IRS particularly effective in inhibiting TLR9 dependent cell stimulation is referred to as “TLR9 class” IRS.

In some embodiments, an IRS may comprise a sequence of the formula: X₁GGGGX₂X₃ (SEQ ID NO:1) wherein X₁, X₂, and X₃ are nucleotides, provided that if X₁=C or A, then X₂X₃ is not AA. In some embodiments, an IRS may comprise a sequence of the formula SEQ ID NO:1 wherein X₁ is C or A. In some embodiments, an IRS may comprise a sequence of the formula: X₁GGGGX₂X₃ (SEQ ID NO:2) wherein X₁, X₂, and X₃ are nucleotides, provided that if X₁=C or A, then X₂X₃ is not AA, and wherein X₁ is C or A.

In some embodiments, an IRS may comprise a sequence of the formula: GGNₙX₁GGGGX₂X₃ (SEQ ID NO:3), wherein n is an integer from 1 to about 100 (preferably from 1 to about 20), each N is a nucleotide, and X₁, X₂, and X₃ are nucleotides, provided that if X₁=C or A, then X₂X₃ is not AA. In some embodiments, an IRS may comprise a sequence of the formula SEQ ID NO:3 wherein X₁ is C or A.

In some embodiments, an IRS may comprise a sequence of the formula: N₁TCCNⱼ(GG)ₖNₘX₁GGGGX₂X₃ (SEQ ID NO: 4), wherein each N is a nucleotide, wherein i is an integer from 1 to about 50, wherein j is an integer from 1 to about 50, k is 0 or 1, m is an integer from 1 to about 20, and X₁, X₂, and X₃ are nucleotides, provided that if X₁=C or A, then X₂X₃ is not AA. In some embodiments, an IRS may comprise a sequence of the formula SEQ ID NO:4 wherein X₁ is C or A.

In some embodiments, an IRS may comprise a sequence of the formula: X₁X₂X₃GGGGAA (SEQ ID NO:5), wherein X₁, X₂, and X₃ are nucleotides, provided that if X₃=C or A, then X₁X₂ is not GG.

Examples of oligonucleotide sequences comprising SEQ ID NO:1, 2, 3, 4, or 5 include the following sequences:

(SEQ ID NO: 10 (C827))
5′-TCCTAACGGGGAAGT-3′;
(SEQ ID NO: 11 (C828))
5′-TCCTAAGGGGGAAGT-3′;
(SEQ ID NO: 12 (C841))
5′-TCCTAACGGGGTTGT-3′;
(SEQ ID NO: 13 (C842))
5′-TCCTAACGGGGCTGT-3′;
(SEQ ID NO: 14 (C843))
5′-TCCTCAAGGGGCTGT-3′;
(SEQ ID NO: 15 (C844))
5′-TCCTCAAGGGGTTGT-3′;
(SEQ ID NO: 16 (C845))
5′-TCCTCATGGGGTTGT-3′;
(SEQ ID NO: 17 (C869))
5′-TCCTGGAGGGGTTGT-3′;
(SEQ ID NO: 18 (C870))
5′-TCCTGGAGGGGCTGT-3′;
(SEQ ID NO: 19 (C871))
5′-TCCTGGAGGGGCCAT-3′;
(SEQ ID NO: 20 (C872))
5′-TCCTGGAGGGGTCAT-3′;
(SEQ ID NO: 21 (C873))
5′-TCCGGAAGGGGAAGT-3′;
(SEQ ID NO: 22 (C874))
5′-TCCGGAAGGGGTTGT-3′;
(SEQ ID NO: 74 (C983))
5′-TGC HEG TGG AGG GGT TGT-3′;
(SEQ ID NO: 75 (C984))
5′-TGC TEG TGG AGG GGT TGT-3′;
(SEQ ID NO: 76 (C985))
5′-TGC ddd TGG AGG GGT TGT-3′;
(SEQ ID NO: 77 (C986))
5′-GC TCC TGG AGG GGT TGT-3′;
(SEQ ID NO: 78 (C987))
5′-C TCC TGG AGG GGT TGT-3′;
(SEQ ID NO: 79 (C988))
5′-AAA TCC TGG AGG GGT TGT-3′;
(SEQ ID NO: 80 (C989))
5′-TCC TGG dGG GGT TGT-3′;
(SEQ ID NO: 81 (C990))
5′-TCC TGG ddG GGG TTG T-3′;
and
(SEQ ID NO: 82 (C991))
5′-TGC TCC TGG AGG GGT TGT HEG HEG-3′,

wherein “d” refers to abasic nucleotides (i.e., lacking a nucleotide base, but having the sugar and phosphate portions).

In some embodiments, an IRS may comprise a sequence of any of SEQ ID NO:1, 2, 3, 4, or 5, wherein at least one G is replaced by 7-deaza-dG. For example, in some embodiments, the IRS may comprise the sequence 5′-TCCTGGAGZ′GGTTGT-3′ (Z′=7-deaza-dG; SEQ ID NO:23 (C920)).

IRPs comprising SEQ ID NO:1, 2, 3, 4, or 5 or an IRP comprising SEQ ID NO:1, 2, 3, 4, or 5, wherein at least one G is replaced by 7-deaza-dG are particularly effective in inhibiting TLR9 dependent cell stimulation. Other IRS sequences which are also effective in inhibiting TLR9 dependent cell signalling include the following:

(SEQ ID NO: 24 (C533))
5′-TGACTGTAGGCGGGGAAGATGA-3′;
(SEQ ID NO: 25 (C707))
5′-GAGCAAGCTGGACCTTCCAT-3′;
and
(Z′ = 7-deaza-dG; SEQ ID NO: 26 (C891))
5′-CCTCAAGCTTGAGZ'GG-3′.

As shown herein, some IRS are particularly effective in inhibiting TLR7/8 dependent cell stimulation. Accordingly, IRS with this activity are referred to as “TLR7/8 class” IRS. For example, an oligonucleotide comprising the sequence 5′-TGCTTGCAAGCTTGCAAGCA-3′ (SEQ ID NO: 27 (C661)) inhibits TLR7/8 dependent cell stimulation.

In some embodiments, an IRS comprises a fragment of SEQ ID NO:27 (C661) and includes at least a 10 base palindromic portion thereof. For example, such sequences include the following sequences:

| | |
|---|---|
| 5′-TGCTTGCAAGCTTGCAAG-3′; | (SEQ ID NO: 28 (C921)) |
| 5′-TGCTTGCAAGCTTGCA-3′; | (SEQ ID NO: 29 (C922)) |
| 5′-GCTTGCAAGCTTGCAAGCA-3′; | (SEQ ID NO: 30 (C935)) |
| 5′-CTTGCAAGCTTGCAAGCA-3′; | (SEQ ID NO: 31 (C936)) |
| and | |
| 5′-TTGCAAGCTTGCAAGCA-3′. | (SEQ ID NO: 32 (C937)) |

In some embodiments, the IRP consists of SEQ ID NO:27 (C661), or a fragment thereof. In some embodiments, an IRP consists of a fragment of SEQ ID NO:27 (C661) and includes at least a 10 base palindromic portion thereof.

In some embodiments, an IRP effective in inhibiting TLR7/8 dependent cell stimulation consists of the sequence 5′-TGCNₘ-3′, where N is a nucleotide, m is an integer from 5 to about 50 and wherein the sequence N₁-Nₘ comprises at least one GC dinucleotide. In some embodiments, such an IRP consists of the sequence 5′-TGCNₘA-3′, the sequence 5′-TGCNₘCA-3′ or the sequence 5′-TGCNₘGCA-3′. For example, in some embodiments, the IRP may consist of the following sequences:

| | |
|---|---|
| 5′-TGCTTGCAAGCTAGCAAGCA-3′; | (SEQ ID NO: 33 (C917)) |
| 5′-TGCTTGCAAGCTTGCTAGCA-3′; | (SEQ ID NO: 34 (C918)) |
| 5′-TGCTTGACAGCTTGACAGCA-3′; | (SEQ ID NO: 35 (C932)) |
| 5′-TGCTTAGCAGCTATGCAGCA-3′; | (SEQ ID NO: 36 (C933)) |
| or | |
| 5′-TGCAAGCAAGCTAGCAAGCA-3′. | (SEQ ID NO: 37 (C934)) |

Other IRS sequences which are also effective in inhibiting TLR7/8 dependent cell signalling include the following:

(SEQ ID NO: 38 (C793))
5′-TGCAAGCTTGCAAGCTTG CAA GCT T-3′;
(SEQ ID NO: 39 (C794))
5′-TGCTGCAAGCTTGCAGAT GAT-3′;
(SEQ ID NO: 40 (C919))
5′-TGCTTGCAAGCTTGCAAGC-3′;
(SEQ ID NO: 41 (C923))
5′-TGCAAGCTTGCAAGCTTGCAAT-3′;
(SEQ ID NO: 42 (C930))
5′-TGCTTGCAAGCTTG-3′;
(SEQ ID NO: 43 (C938))
5′-AGCTTGCAAGCTTGCAAGCA-3′;
(SEQ ID NO: 44 (C939))
5′-TACTTGCAAGCTTGCAAGCA-3′;
(SEQ ID NO: 45 (C940))
5′-TGATTGCAAGCTTGCAAGCA-3′;
(SEQ ID NO: 46 (C941))
5′-AAATTGCAAGCTTGCAAGCA-3′;
(SEQ ID NO: 47 (C945))
5′-TGCTGGAGGGGTTGT-3′;
(SEQ ID NO: 48 (C951))
5′-AAATTGACAGCTTGACAGCA-3′;
(SEQ ID NO: 49 (C959))
5′-TGATTGACAGCTTGACAGCA-3′;
(SEQ ID NO: 50 (C960))
5′-TGATTGACAGATTGACAGCA-3′;
and
(SEQ ID NO: 51 (C961))
5′-TGATTGACAGATTGACAGAC-3′.

Another class of IRS include those which are particularly effective in inhibiting both TLR7/8 and TLR9 dependent cell stimulation. Accordingly, IRS with this activity are referred to as “TLR7/8/9 class” IRS. In some instances, a combination of a TLR7/8 class IRS with a TLR9 class IRS results in an IRS of the TLR7/8/9 class.

The TLR7/8/9 class of IRS include those comprising the sequence TGCNₘTCCTGGAGGGGTTGT-3′ (SEQ ID NO:6) where each N is a nucleotide and m is an integer from 0 to about 100, in some instances from 0 to about 50, preferably from 0 to about 20.

In some embodiments, an IRS comprises SEQ ID NO:6, wherein the sequence N₁-Nₘ comprises a fragment of the sequence 5′-TTGACAGCTTGACAGCA-3′ (SEQ ID NO:7). A fragment of SEQ ID NO:7 is any portion of that sequence, for example, TTGAC or GCTTGA. In some embodiments, the fragment of SEQ ID NO:7 is from the 5′ end of SEQ ID NO:7, including, for example, TTGAC or TTG.

In some embodiments, the IRS comprises the sequence 5′-TGCRRZNYY-3′ (SEQ ID NO:8), wherein Z is any nucleotide except C, wherein N is any nucleotide, wherein when Z is not G or inosine, N is guanosine or inosine. In other embodiments, the IRS comprises the sequence 5′-TGCRRZNpoly(Pyrimidine)-3′ (SEQ ID NO:9), wherein Z is any nucleotide except C, wherein N is any nucleotide, wherein when Z is not G or inosine, N is guanosine or inosine.

Examples of IRS sequences which are also effective in inhibiting TLR7/8/9 dependent cell signalling include the following:

(SEQ ID NO: 52 (C954))
5′-TGCTCCTGGAGGGGTTGT-3′;
(SEQ ID NO: 53 (C956))
5′-TGCTTGTCCTGGAGGGGTTGT-3′;
(SEQ ID NO: 54 (C957))
5′-TGCTTGACATCCTGGAGGGGTTGT-3′;
(SEQ ID NO: 55 (C962))
5′-TGCTTGACAGCTTGACAGTCCTGGAGGGGTTGT-3′;
(SEQ ID NO: 56 (C963))
5′-TGCTTGACAGCTTGATCCTGGAGGGGTTGT-3′;
(SEQ ID NO: 57 (C964))
5′-TGCTTGACAGCTTCCTGGAGGGGTTGT-3′;
(SEQ ID NO: 58 (C965))
5′-TGCTTGACAGCTTGCTCCTGGAGGGGTTGT-3′;
(SEQ ID NO: 59 (C966))
5′-TGCTTGACAGCTTGCTTGTCCTGGAGGGGTTGT-3′;
(SEQ ID NO: 60 (C967))
5′-TGCTTGACAGCTTGACAGCATCCTGGAGGGGTTGT-3′;
(SEQ ID NO: 61 (C968))
5′-TGCTTGACAGCTTGACAGCATCCTGGAGGGGTTGT-3′;
(SEQ ID NO: 62 (C969))
5′-TGCTTGACAGCTTGACAGCATCCTGGAGGGGT-3′;
(SEQ ID NO: 63 (C970))
5′-TGCTTGACAGCTTGACAGCATCCTGGAGGGG-3′;
(SEQ ID NO: 64 (C971))
5′-TGCTTGCAAGCTTGCTCCTGGAGGGGTTGT-3′;
(SEQ ID NO: 65 (C972))
5′-TGCTTGCAAGCTTCCTGGAGGGGTTGT-3′;
and
(SEQ ID NO: 66 (C908))
5′-TGCTTGCAAGCTTGCAAGCATCCTGGAGGGGTTGT-3′.

As described herein, some IRPs are particularly effective in suppressing TLR9 dependent cell responses. Such IRPs include, but are not limited to, SEQ ID NO:24 (C533); SEQ ID NO:25 (C707); SEQ ID NO:86 (1019); SEQ ID NO:91 (C891); SEQ ID NO:10 (C827); SEQ ID NO:11 (C828); SEQ ID NO:12 (C841); SEQ ID NO:13 (C842); SEQ ID NO:14 (C843); SEQ ID NO:15 (C844); SEQ ID NO:16 (C845); SEQ ID NO:17 (C869); SEQ ID NO:18 (C870); SEQ ID NO:19 (871); SEQ ID NO:20 (C872); SEQ ID NO:21 (C873); SEQ ID NO:22 (C874); SEQ ID NO:23 (C920), and SEQ ID NO:66 (C908). As described herein, some IRPs are particularly effective in suppressing TLR7/8 dependent cell responses. Such IRPs include, but are not limited to, SEQ ID NO:17 (C869); SEQ ID NO:23 (C920); SEQ ID NO:27 (C661); SEQ ID NO:38 (C793); SEQ ID NO:29 (C794); SEQ ID NO:33 (C917); SEQ ID NO:34 (C918); SEQ ID NO:40 (C919); SEQ ID NO:28 (C921); SEQ ID NO:29 (C922); SEQ ID NO:41 (C923), and SEQ ID NO:66 (C908).

Non-immunostimulatory polynucleotides include a polynucleotide with a particular activity but which are non-immunostimulatory, for example aptamers or antisense molecules. Aptamers are of use as targeting ligands for delivery of imaging and/or therapeutic reagents to particular cells or tissues. Aptamers are high affinity, high specificity RNA or DNA-based ligands produced by in vitro selection experiments (SELEX: systematic evolution of ligands by exponential enrichment). Aptamers are generated from random sequences of 20 to 30 nucleotides, selectively screened by absorption to molecular antigens or cells, and enriched to purify specific high affinity binding ligands.

A NISC polynucleotide may be single stranded or double stranded DNA, as well as single or double-stranded RNA or other modified polynucleotides. A NISC polynucleotide may be linear, may be circular or include circular portions and/or may include a hairpin loop. A NISC polynucleotide may contain naturally-occurring or modified, non-naturally occurring bases, and may contain modified sugar, phosphate, and/or termini. Various such modifications are described herein.

The heterocyclic bases, or nucleic acid bases, which are incorporated in the NISC polynucleotide can be the naturally-occurring principal purine and pyrimidine bases, (namely uracil, thymine, cytosine, adenine and guanine, as mentioned above), as well as naturally-occurring and synthetic modifications of said principal bases. Thus, a NISC polynucleotide may include 2′-deoxyuridine and/or 2-amino-2′-deoxyadenosine.

The NISC polynucleotide may comprise at least one modified base. As used herein, the term “modified base” is synonymous with “base analog”, for example, “modified cytosine” is synonymous with “cytosine analog.” Similarly, “modified” nucleosides or nucleotides are herein defined as being synonymous with nucleoside or nucleotide “analogs.” Examples of base modifications include, but are not limited to, addition of an electron-withdrawing moiety to C-5 and/or C-6 of a cytosine of the NISC polynucleotide. Preferably, the electron-withdrawing moiety is a halogen, e.g., 5-bromocytosine, 5-chlorocytosine, 5-fluorocytosine, 5-iodocytosine. Other examples of base modifications include, but are not limited to, addition of an electron-withdrawing moiety to C-5 and/or C-6 of a uracil of the NISC polynucleotide. Preferably, the electron-withdrawing moiety is a halogen. Such modified uracils can include, but are not limited to, 5-bromouracil, 5-chlorouracil, 5-fluorouracil, 5-iodouracil.

Other examples of base modifications include the addition of one or more thiol groups to the base including, but not limited to, 6-thio-guanine, 4-thio-thymine, and 4-thio-uracil. Other examples of base modifications include, but are not limited to, N4-ethylcytosine, 7-deazaguanine, and 5-hydroxycytosine. See, for example, Kandimalla et al. (2001) Bioorg. Med. Chem. 9:807-813.

The NISC polynucleotide can contain phosphate-modified polynucleotides, some of which are known to stabilize the polynucleotide. Accordingly, some embodiments includes stabilized NISC polynucleotides. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, oligonucleotides of the present invention comprise only phosphorothioate backbones. In some embodiments, polynucleotides of the present invention comprise only phosphodiester backbones. In some embodiments, a NISC polynucleotide may comprise a combination of phosphate linkages in the phosphate backbone such as a combination of phosphodiester and phosphorothioate linkages.

NISC polynucleotides used in the invention can comprise one or more ribonucleotides (containing ribose as the only or principal sugar component), deoxyribonucleotides (containing deoxyribose as the principal sugar component), or, as is known in the art, modified sugars or sugar analogs can be incorporated in the NISC polynucleotide. Thus, in addition to ribose and deoxyribose, the sugar moiety can be pentose, deoxypentose, hexose, deoxyhexose, glucose, arabinose, xylose, lyxose, and a sugar “analog” cyclopentyl group. The sugar can be in pyranosyl or in a furanosyl form. In the polynucleotide, the sugar moiety is preferably the furanoside of ribose, deoxyribose, arabinose or 2′-0-alkylribose, and the sugar can be attached to the respective heterocyclic bases either in a or β anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification in the polynucleotide includes, but is not limited to, 2′-O-methyl-uridine and 2′-O-methyl-cytidine. The preparation of these sugars or sugar analogs and the respective “nucleosides” wherein such sugars or analogs are attached to a heterocyclic base (nucleic acid base) per se is known, and need not be described here, except to the extent such preparation can pertain to any specific example. Sugar modifications may also be made and combined with any phosphate modification in the preparation of an NISC polynucleotide.

The NISC polynucleotide can be synthesized using techniques and nucleic acid synthesis equipment which are well known in the art including, but not limited to, enzymatic methods, chemical methods, and the degradation of larger oligonucleotide sequences. See, for example, Ausubel et al. (1987); and Sambrook et al. (1989). When assembled enzymatically, the individual units can be ligated, for example, with a ligase such as T4 DNA or RNA ligase. U.S. Pat. No. 5,124,246. Polynucleotide degradation can be accomplished through the exposure of an oligonucleotide to a nuclease, as exemplified in U.S. Pat. No. 4,650,675.

The NISC polynucleotide can also be isolated using conventional polynucleotide isolation procedures. Such procedures include, but are not limited to, hybridization of probes to genomic or cDNA libraries to detect shared nucleotide sequences, antibody screening of expression libraries to detect shared structural features and synthesis of particular native sequences by the polymerase chain reaction.

Circular NISC polynucleotides can be isolated, synthesized through recombinant methods, or chemically synthesized. Where the circular NISC polynucleotide is obtained through isolation or through recombinant methods, the NISC polynucleotide will preferably be a plasmid. The chemical synthesis of smaller circular oligonucleotides can be performed using any method described in the literature. See, for instance, Gao et al. (1995) Nucleic Acids Res. 23:2025-2029; and Wang et al. (1994) Nucleic Acids Res. 22:2326-2333.

The techniques for making polynucleotides and modified polynucleotides are known in the art. Naturally occurring DNA or RNA, containing phosphodiester linkages, is generally synthesized by sequentially coupling the appropriate nucleoside phosphoramidite to the 5′-hydroxy group of the growing oligonucleotide attached to a solid support at the 3′-end, followed by oxidation of the intermediate phosphite triester to a phosphate triester. Once the desired polynucleotide sequence has been synthesized, the polynucleotide is removed from the support, the phosphate triester groups are deprotected to phosphate diesters and the nucleoside bases are deprotected using aqueous ammonia or other bases. See, for example, Beaucage (1993) “Oligodeoxyribonucleotide Synthesis” in Protocols for Oligonucleotides and Analogs, Synthesis and Properties (Agrawal, ed.) Humana Press, Totowa, N.J.; Warner et al. (1984) DNA 3:401 and U.S. Pat. No. 4,458,066.

Synthesis of polynucleotides containing modified phosphate linkages or non-phosphate linkages is also known in the art. For a review, see Matteucci (1997) “Oligonucleotide Analogs: an Overview” in Oligonucleotides as Therapeutic Agents, (D. J. Chadwick and G. Cardew, ed.) John Wiley and Sons, New York, N.Y. The phosphorous derivative (or modified phosphate group) which can be attached to the sugar or sugar analog moiety in the polynucleotides of the present invention can be a monophosphate, diphosphate, triphosphate, alkylphosphonate, phosphorothioate, phosphorodithioate, phosphoramidate or the like. The preparation of the above-noted phosphate analogs, and their incorporation into nucleotides, modified nucleotides and oligonucleotides, per se, is also known and need not be described here in detail. Peyrottes et al. (1996) Nucleic Acids Res. 24:1841-1848; Chaturvedi et al. (1996) Nucleic Acids Res. 24:2318-2323; and Schultz et al. (1996) Nucleic Acids Res. 24:2966-2973. For example, synthesis of phosphorothioate oligonucleotides is similar to that described above for naturally occurring oligonucleotides except that the oxidation step is replaced by a sulfurization step (Zon (1993) “Oligonucleoside Phosphorothioates” in Protocols for Oligonucleotides and Analogs, Synthesis and Properties (Agrawal, ed.) Humana Press, pp. 165-190). Similarly the synthesis of other phosphate analogs, such as phosphotriester (Miller et al. (1971) JACS 93:6657-6665), non-bridging phosphoramidates (Jager et al. (1988) Biochem. 27:7247-7246), N3′ to P5′ phosphoramidiates (Nelson et al. (1997) JOC 62:7278-7287) and phosphorodithioates (U.S. Pat. No. 5,453,496) has also been described. Other non-phosphorous based modified oligonucleotides can also be used (Stirchak et al. (1989) Nucleic Acids Res. 17:6129-6141).

Those skilled in the art will recognize that a large number of “synthetic” non-natural nucleosides comprising various heterocyclic bases and various sugar moieties (and sugar analogs) are available in the art, and that as long as other criteria of the present invention are satisfied, the NISC polynucleotide can include one or several heterocyclic bases other than the principal five base components of naturally-occurring nucleic acids. Preferably, however, the heterocyclic base in the NISC polynucleotide includes, but is not limited to, uracil-5-yl, cytosin-5-yl, adenin-7-yl, adenin-8-yl, guanin-7-yl, guanin-8-yl, 4-aminopyrrolo[2,3-d]pyrimidin-5-yl, 2-amino-4-oxopyrolo[2,3-d]pyrimidin-5-yl, 2-amino-4-oxopyrrolo[2,3-d]pyrimidin-3-yl groups, where the purines are attached to the sugar moiety of the NISC polynucleotide via the 9-position, the pyrimidines via the 1-position, the pyrrolopyrimidines via the 7-position and the pyrazolopyrimidines via the 1-position.

The preparation of base-modified nucleosides, and the synthesis of modified polynucleotides using said base-modified nucleosides as precursors, has been described, for example, in U.S. Pat. Nos. 4,910,300, 4,948,882, and 5,093,232. These base-modified nucleosides have been designed so that they can be incorporated by chemical synthesis into either terminal or internal positions of an oligonucleotide. Such base-modified nucleosides, present at either terminal or internal positions of an oligonucleotide, can serve as sites for attachment of a peptide. Nucleosides modified in their sugar moiety have also been described (including, but not limited to, e.g., U.S. Pat. Nos. 4,849,513, 5,015,733, 5,118,800, 5,118,802) and can be used similarly.

In some embodiments, a NISC polynucleotide is less than about any of the following lengths (in bases or base pairs): 10,000; 5,000; 2500; 2000; 1500; 1250; 1000; 750; 500; 300; 250; 200; 175; 150; 125; 100; 75; 60; 50; 40; 30; 25; 20; 15; 14; 13; 12; 11; 10; 9; 8; 7; 6; 5; 4. In some embodiments, a NISC polynucleotide is greater than about any of the following lengths (in bases or base pairs): 4; 5; 6, 7, 8, 9, 10; 11; 12; 13; 14; 15; 20; 25; 30; 40; 50; 60; 75; 100; 125; 150; 175; 200; 250; 300; 350; 400; 500; 750; 1000; 2000; 5000; 7500; 10000; 20000; 50000. Alternately, the NISC polynucleotide can be any of a range of sizes having an upper limit of 10,000; 5,000; 2500; 2000; 1500; 1250; 1000; 750; 500; 300; 250; 200; 175; 150; 125; 100; 75; 60; 50; 40; 30; 25; 20; 15; 14; 13; 12; 11; 10; 9; 8; 7; 6; 5; 4 and an independently selected lower limit of 4; 5; 6; 7; 8; 9; 10; 11; 12; 13; 14; 15; 20; 25; 30; 40; 50; 60; 75; 100; 125; 150; 175; 200; 250; 300; 350; 400; 500; 750; 1000; 2000; 5000; 7500, wherein the lower limit is less than the upper limit. In some embodiments, a NISC polynucleotide is preferably about 200 or less bases in length.

The invention also provides methods of making the NISC polynucleotides described herein. The methods may be any of those described herein. For example, the method could be synthesizing the NISC polynucleotide (for example, using solid state synthesis) and may further comprise any purification step(s). Methods of purification are known in the art.

In certain embodiments, the invention is directed to non-stimulatory conjugates comprising immunoregulatory compounds (IRCs) as the polynucleotide component. Such IRCs have immunoregulatory activity and comprise a nucleic acid moiety comprising an IRS. IRCs contain one or more nucleic acid moieties and one or more non-nucleic acid spacer moieties. Compounds conforming to a variety of structural formulas are contemplated for use as IRCs in the NISCs, including the core structures described in formulas I-VII, below. Formulas I-III show core sequences for “linear IRCs.” Formulas IV-VI show core sequences for “branched IRCs.” Formula VII shows a core structure for “single-spacer IRCs.”

In each formula provided herein, “N” designates a nucleic acid moiety (oriented in either a 5′→3′ or 3′→5′ orientation) and “S” designates a non-nucleic acid spacer moiety. A dash (“-”) designates a covalent bond between a nucleic acid moiety and a non-nucleic acid spacer moiety. A double dash (“--”) designates covalent bonds between a non-nucleic acid spacer moiety and at least 2 nucleic acid moieties. A triple dash (“---”) designates covalent bonds between a non-nucleic acid spacer moiety and multiple (i.e., at least 3) nucleic acid moieties. Subscripts are used to designate differently positioned nucleic acid or non-nucleic acid spacer moieties. However, the use of subscripts to distinguish different nucleic acid moieties is not intended to indicate that the moieties necessarily have a different structure or sequence. Similarly, the use of subscripts to distinguish different spacer moieties is not intended to indicate that the moieties necessarily have different structures. For example, in formula II, infra, the nucleic acid moieties designated N₁ and N₂ can have the same or different sequences, and the spacer moieties designated S₁ and S₂ can have the same or different structures. Further, it is contemplated that additional chemical moieties (e.g., phosphate, mononucleotide, additional nucleic acid moieties, alkyl, amino, thio or disulfide groups or linking groups, and/or spacer moieties) may be covalently bound at the termini of the core structures.

Linear IRCs have structures in which the non-nucleic acid spacer moieties in the core structure are covalently bound to no more than two nucleic acid moieties. Exemplary linear IRCs conform to the following formulas:

N₁-S₁-N₂ (I)

N₁-S₁-N₂-S₂-N₃ (II).

N₁-S₁-N₂-S₂-[Nᵥ-Sᵥ]_{A} (III)

where A is an integer between 1 and about 100 and [Nᵥ-S_{z}] indicates A additional iterations of nucleic acid moieties conjugated to non-nucleic acid spacer moieties. The subscript “v” indicates that N and S are independently selected in each iteration of “[Nᵥ-Sᵥ].” “A” is sometimes between 1 and about 10, sometimes between 1 and 3, sometimes exactly 1, 2, 3, 4 or 5. In some embodiments, A is an integer in a range defined by a lower limit of 1, 2, 3, 4, or 5, and an independently selected upper limit of 10, 20, 50 or 100 (e.g., between 3 and 10).

Exemplary linear IRCs include:

N₁-HEG-N₂—OH (Ia)

N₁-HEG-N₁—PO₄ (Ib)

N₁-HEG-N₂-HEG (Ic)

HEG-N₁-HEG-N₁-HEG (Id)

N₁-HEG-N₂-HEG-N₁ (Ie)

N₁-HEG-N₂-(HEG)₄-N₃ (If)

(N₁)₂-glycerol-N₁-HEG-N₁ (Ig)

PO₄—N₁-HEG-N₂ (Ih)

N₁-(HEG)₁₅-T (Ii)

(N₁-HEG)₂-glycerol-HEG-N₂ (Ij)

N₁-HEG-T-HEG-T (Ik)

Wherein HEG refers to hexa-(ethylene glycol). TEG refers to tetra-(ethylene glycol).

Preferred linear IRCs include:

(SEQ ID NO: 67 (C907, C661-HEG-C869)
5′-TGCTTGCAAGCTTGCAAGCA-HEG-TCCTGGAGGGGTTGT-3′;
(SEQ ID NO: 68 (C913, C917-HEG-C869)
5′-TGCTTGCAAGCTAGCAAGCA-HEG-TCCTGGAGGGGTTGT-3′;
(SEQ ID NO: 69 (C914, C918-HEG-C869)
5′-TGCTTGCAAGCTTGCTAGCA-HEG-TCCTGGAGGGGTTGT-3′;
(SEQ ID NO: 70 (C916, C661-HEG-C920)
5′-TGCTTGCAAGCTTGCTAGCA-HEG-TCCTGGAGZGGTTGT-3′;
and
(SEQ ID NO: 71 (C928, C869-HEG-C661)
5′-TCCTGGAGGGGTTGT-HEG-TGCTTGCAAGCTTGCAAGCA-3′.

Branched IRCs comprise a multivalent spacer moiety (Sₚ) covalently bound to at least three (3) nucleic acid moieties. Exemplary branched IRCs are described according to the following formulas

[Nᵥ]_{A}---Sₚ (IV)

[Sᵥ-Nᵥ]_{A}---Sₚ (V)

(S₁-N₁)-Sₚ--(Nᵥ)_{A} (VI)

where Sₚ is a multivalent spacer covalently bonded to the quantity “A” independently selected nucleic acid moieties Nᵥ, Sᵥ-Nᵥ (which comprises a spacer moiety covalently bound to a nucleic acid moiety). For formulas IV and V, A is at least 3. In various embodiments of formulas IV and V, A is an integer between 3 and 100 (inclusive), although A may be an integer in a range defined by a lower limit of about 3, 5, 10, 50, or 100 and an independently selected upper limit of about 5, 7, 10, 50, 100, 150, 200, 250, or 500, or alternately A may be greater than 500. For formula VI, A is at least 2, an integer in a range defined by a lower limit of 2, 5, 10, 50, or 100 and an independently selected upper limit of 5, 10, 50, 100, 150, 200, 250, or 500, or greater than 500.

Exemplary branched IRCs include:

(N₁)₂-glycerol-N₁ (IVa)

(N₂-HEG)₂-glycerol-N₁ (IVb)

(N₁-HEG-N₂)₂-glycerol-N₁ (IVc)

[(N₁)₂-glycerol-N₁]₂-glycerol-N₁ (IVd)

Preferred branched IRCs include (5′-N₁-3′-HEG)₂-glycerol-HEG-5′-N₁-3′ and (5′-N₁-3′-HEG)₂-glycerol-HEG-5′-N₁′.

Single spacer IRCs comprise a structure in which there is a single nucleic acid moiety covalently conjugated to a single spacer moiety, i.e.,

N₁-S₁ (VII)

In a preferred embodiment S₁ has the structure of a multimer comprising smaller units (e.g., HEG, TEG, glycerol, 1′2′-dideoxyribose, C2 alkyl-C12 alkyl subunits, and the like), typically connected by an ester linkage (e.g., phosphodiester or phosphorothioate ester), e.g., as described infra. See, e.g., formula VIIa, infra. The multimer can be heteromeric or homomeric. In one embodiment, the spacer is a heteromer of monomeric units (e.g., HEG, TEG, glycerol, 1′2′-dideoxyribose, C2 alkyl to C12 alkyl linkers, and the like) linked by an ester linkage (e.g., phosphodiester or phosphorothioate ester). See, e.g., formula VIIb, infra.

Exemplary single spacer IRCs include:

N₁-(HEG)₁₅ (VIIa)

N₁-HEG-propyl-HEG-propyl-HEG (VIIb)

In certain embodiments, the terminal structures of the IRC are covalently joined (e.g., nucleic acid moiety-to-nucleic acid moiety; spacer moiety-to-spacer moiety, or nucleic acid moiety-to-spacer moiety), resulting in a circular conformation.

IRCs for use in the NISCs of the invention include at least one nucleic acid moiety. The term “nucleic acid moiety,” as used herein, refers to a nucleotide monomer (i.e., a mononucleotide) or polymer (i.e., comprising at least 2 contiguous nucleotides). As used herein, a nucleotide comprises (1) a purine or pyrimidine base linked to a sugar that is in an ester linkage to a phosphate group, or (2) an analog in which the base and/or sugar and/or phosphate ester are replaced by analogs, e.g., as described infra. In an IRC comprising more than one nucleic acid moiety, the nucleic acid moieties may be the same or different.

Nucleic acid moieties used in IRCs incorporated in the NISCs may comprise any of the IRS sequences disclosed herein, and may additionally be sequences of six base pairs or less. It is contemplated that in an IRC comprising multiple nucleic acid moieties, the nucleic acid moieties can be the same or different lengths. In certain embodiments where the IRC comprises more than one nucleic acid moiety, only one of the moieties need comprise the IRS.

It is contemplated that in a IRC comprising multiple nucleic acid moieties, the nucleic acid moieties can be the same or different. Accordingly, in various embodiments, IRCs incorporated into the NISCs comprise (a) nucleic acid moieties with the same sequence, (b) more than one iteration of a nucleic acid moiety, or (c) two or more different nucleic acid moieties. Additionally, a single nucleic acid moiety may comprise more than one IRS, which may be adjacent, overlapping, or separated by additional nucleotide bases within the nucleic acid moiety.

As described herein, some IRPs are particularly effective in suppressing TLR9 dependent cell responses and some IRPs are particularly effective in suppressing TLR7/8 dependent cell responses. Since an IRC may comprise more than one IRP, IRPs with various activities can be combined to create an IRC with a particular activity for a particular use.

In some instances, the combination of two IRPs in an IRC leads to an immunoregulatory activity of the IRC different from either of the IRPs alone. Whatever the combination, the oligonucleotide in the NISC stimulates little of no APC/DC activation or maturation. For example, IRC SEQ ID NO:68 (C913 contains IRP SEQ ID NO:33 (C917) linked to IRP SEQ ID NO:17 (C869) through a HEG moiety. IRP SEQ ID NO:33 (C917) inhibits TLR-7/8 dependent cell responses but not TLR-9 dependent cell responses. IRP SEQ ID NO:17 (C869) have greater inhibitory activity for TLR-9 dependent cell responses than for TLR-7/8 dependent cell responses. The IRC SEQ ID NO:68 (C913) however is very active in inhibiting both TLR-7/8 dependent cell responses and TLR-9 dependent cell responses. The same is also true for IRC SEQ ID NO:69 (C914) and its component IRPs SEQ ID NO:34 (C918) and SEQ ID NO:17 (C869).

The IRCs comprise one or more non-nucleic acid spacer moieties covalently bound to the nucleic acid moieties. For convenience, non-nucleic acid spacer moieties are sometimes referred to herein simply as “spacers” or “spacer moieties.” Spacers are generally of molecular weight about 50 to about 50,000, typically from about 75 to about 5000, most often from about 75 to about 500, which are covalently bound, in various embodiments, to one, two, three, or more than three nucleic acid moieties. A variety of agents are suitable for connecting nucleic acid moieties. For example, a variety of compounds referred to in the scientific literature as “non-nucleic acid linkers,” “non-nucleotidic linkers,” or “valency platform molecules” may be used as spacers in an IRC. In certain embodiments, a spacer comprises multiple covalently connected subunits and may have a homopolymeric or heteropolymeric structure. It will be appreciated that mononucleotides and polynucleotides are not included in the definition of non-nucleic acid spacers, without which exclusion there would be no difference between nucleic acid moiety and an adjacent non-nucleic acid spacer moiety.

In certain embodiments, a spacer may comprise one or more abasic nucleotides (i.e., lacking a nucleotide base, but having the sugar and phosphate portions) designated herein as “d”. Exemplary abasic nucleotides include 1′2′-dideoxyribose, 1′-deoxyribose, 1′-deoxyarabinose and polymers thereof.

Other suitable spacers comprise optionally substituted alkyl, optionally substituted polyglycol, optionally substituted polyamine, optionally substituted polyalcohol, optionally substituted polyamide, optionally substituted polyether, optionally substituted polyimine, optionally substituted polyphosphodiester (such as poly(1-phospho-3-propanol), and the like. Optional substituents include alcohol, alkoxy (such as methoxy, ethoxy, and propoxy), straight or branched chain alkyl (such as C1-C12 alkyl), amine, aminoalkyl (such as amino C1-C12 alkyl), phosphoramidite, phosphate, thiophosphate, hydrazide, hydrazine, halogen, (such as F, Cl, Br, or I), amide, alkylamide (such as amide C1-C12 alkyl), carboxylic acid, carboxylic ester, carboxylic anhydride, carboxylic acid halide, sulfonyl halide, imidate ester, isocyanate, isothiocyanate, haloformate, carbodiimide adduct, aldehydes, ketone, sulfhydryl, haloacetyl, alkyl halide, alkyl sulfonate, NR1R2 wherein R1R2 is —C(═O)CH═CHC(═O) (maleimide), thioether, cyano, sugar (such as mannose, galactose, and glucose), α,β-unsaturated carbonyl, alkyl mercurial, α,β-unsaturated sulfone.

Suitable spacers may comprise polycyclic molecules, such as those containing phenyl or cyclohexyl rings. The spacer may be a polyether such as polyphosphopropanediol, polyethyleneglycol, polypropylene glycol, a bifunctional polycyclic molecule such as a bifunctional pentalene, indene, naphthalene, azulene, heptalene, biphenylene, asymindacene, sym-indacene, acenaphthylene, fluorene, phenalene, phenanthrene, anthracene, fluoranthene, acephenathrylene, aceanthrylene, triphenylene, pyrene, chrysene, naphthacene, thianthrene, isobenzofuran, chromene, xanthene, phenoxathiin, which may be substituted or modified, or a combination of the polyethers and the polycyclic molecules. The polycyclic molecule may be substituted or polysubstituted with C1-C5 alkyl, C6 alkyl, alkenyl, hydroxyalkyl, halogen or haloalkyl group. Nitrogen-containing polyheterocyclic molecules (e.g., indolizine) are typically not suitable spacers. The spacer may also be a polyalcohol, such as glycerol or pentaerythritol. In one embodiment, the spacer comprises 1-phosphopropane)₃-phosphate or 1-phosphopropane)₄-phosphate (also called tetraphosphopropanediol and pentaphosphopropanediol). In one embodiment, the spacer comprises derivatized 2,2′-ethylenedioxydiethylamine (EDDA).

Specific examples of non-nucleic acid spacers useful in IRCs include “linkers” described by Cload et al. (1991) J. Am. Chem. Soc. 113:6324; Richardson et al. (1991) J. Am. Chem. Soc. 113:5109; Ma et al. (1993) Nucleic Acids Res. 21:2585; Ma et al. (1993) Biochemistry 32:1751; McCurdy et al. (1991) Nucleosides & Nucleotides 10:287; Jaschke et al. (1993) Tetrahedron Lett. 34:301; Ono et al. (1991) Biochemistry 30:9914; and International Publication No. WO 89/02439.

Other suitable spacers include linkers described by Salunkhe et al. (1992) J. Am. Chem. Soc. 114:8768; Nelson et al. (1996) Biochemistry 35:5339-5344; Bartley et al. (1997) Biochemistry 36:14502-511; Dagneaux et al. (1996) Nucleic Acids Res. 24:4506-12; Durand et al. (1990) Nucleic Acids Res. 18:6353-59; Reynolds et al. (1996) Nucleic Acids Res. 24:760-65; Hendry et al. (1994) Biochem. Biophys. Acta 1219:405-12; Altmann et al. (1995) Nucleic Acids Res. 23:4827-35. Still other suitable spacers are described in European Pat. No. EP0313219B1 and U.S. Pat. No. 6,117,657.

Exemplary non-nucleic acid spacers comprise oligo-ethylene glycol (e.g., triethylene glycol, tetraethylene glycol, hexaethylene glycol spacers, and other polymers comprising up to about 10, about 20, about 40, about 50, about 100 or about 200 ethylene glycol units), alkyl spacers (e.g., propyl, butyl, hexyl, and other C2-C12 alkyl spacers, e.g., usually C2-C10 alkyl, most often C2-C6 alkyl), abasic nucleotide spacers, symmetric or asymmetric spacers derived from glycerol, pentaerythritol or 1,3,5-trihydroxycyclohexane (e.g., symmetrical doubler and trebler spacer moieties described herein). Spacers can also comprise heteromeric or homomeric oligomers and polymers of the aforementioned compounds (e.g., linked by an amide, ester, ether, thioether, disulfide, phosphodiester, phosphorothioate, phosphoramidate, phosphotriester, phosphorodithioate, methyl phosphonate or other linkage).

Suitable spacer moieties can contribute charge and/or hydrophobicity to the IRC, contribute favorable pharmacokinetic properties (e.g., improved stability, longer residence time in blood) to the IRC, and/or result in targeting of the IRC to particular cells or organs. Spacer moieties can be selected or modified to tailor the IRC for desired pharmacokinetic properties or suitability for desired modes of administration (e.g., oral administration). It will be appreciated by the reader that, for convenience, a spacer (or spacer component) is sometimes referred to by the chemical name of the compound from which the spacer component is derived (e.g., hexaethylene glycol), with the understanding that the IRC actually comprises the conjugate of the compound and adjacent nucleic acid moieties or other spacer moiety components.

In an IRC comprising more than one spacer moiety, the spacers may be the same or different. Thus, in one embodiment all of the non-nucleic acid spacer moieties in an IRC have the same structure. In one embodiment, an IRC comprises non-nucleic acid spacer moieties with at least 2, at least 3, at least 4, at least 5, or at least 6 or more different structures.

In some contemplated embodiments of the invention, the spacer moiety of an IRC is defined to exclude certain structures. Thus, in some embodiments of the invention, a spacer is other than an abasic nucleotide or polymer of abasic nucleotides. In some embodiments of the invention, a spacer is other than a oligo(ethyleneglycol) (e.g., HEG, TEG and the like) or poly(ethyleneglycol). In some embodiments a spacer is other than a C3 alkyl spacer. In some embodiments, a spacer is other than a polypeptide. Thus, in some embodiments, an immunogenic molecule, e.g., a protein or polypeptide, is not suitable as a component of spacer moieties. However, as discussed infra, it is contemplated that in certain embodiments, an IRC is a “proteinaceous IRC” i.e., comprising a spacer moiety comprising a polypeptide. However, in some embodiments, the spacer moiety is not proteinaceous and/or is not an antigen (i.e., the spacer moiety, if isolated from the IRC, is not an antigen).

Generally, suitable spacer moieties do not render the IRC of which they are a component insoluble in an aqueous solution (e.g., PBS, pH 7.0). Thus, the definition of spacers excludes microcarriers or nanocarriers. In addition, a spacer moiety that has low solubility, such as a dodecyl spacer (solubility<5 mg/ml when measured as dialcohol precursor 1,12-dihydroxydodecane) is not preferred because it can reduce the hydrophilicity and activity of the IRC. Preferably, spacer moieties have solubility much greater than 5 mg/ml (e.g., ≧20 mg/ml, ≧50 mg/ml or ≧100 mg/ml) when measured as dialcohol precursors.

The charge of an IRC may be contributed by phosphate, thiophosphate, or other groups in the nucleic acid moieties as well as groups in non-nucleic acid spacer moieties. In some embodiments of the invention, a non-nucleic acid spacer moiety carries a net charge (e.g., a net positive charge or net negative charge when measured at pH 7). In one useful embodiment, the IRC has a net negative charge. In some embodiments, the negative charge of a spacer moiety in an IRC is increased by derivatizing a spacer subunit described herein to increase its charge. For example, glycerol can be covalently bound to two nucleic acid moieties and the remaining alcohol can be reacted with an activated phosphoramidite, followed by oxidation or sulfurization to form a phosphate or thiophosphate, respectively. In certain embodiments the negative charge contributed by the non-nucleic acid spacer moieties in an IRC (i.e., the sum of the charges when there is more than one spacer) is greater than the negative charge contributed by the nucleic acid moieties of the IRC. Charge can be calculated based on molecular formula, or determined experimentally, e.g., by capillary electrophoresis (Li, ed., 1992, Capillary electrophoresis, Principles, Practice and Application Elsevier Science Publishers, Amsterdam, The Netherlands, pp 202-206).

As is noted supra, suitable spacers can be polymers of smaller non-nucleic acid (e.g., non-nucleotide) compounds, such as those described herein, that are themselves useful as spacers, including compounds commonly referred to as non-nucleotide “linkers.” Such polymers (i.e., “multiunit spacers”) may be heteromeric or homomeric, and often comprise monomeric units (e.g., HEG, TEG, glycerol, 1′2′-dideoxyribose, and the like) linked by an ester linkage (e.g., phosphodiester or phosphorothioate ester). Thus, in one embodiment the spacer comprises a polymeric (e.g., heteropolymeric) structure of non-nucleotide units (e.g., from 2 to about 100 units, alternatively 2 to about 50, e.g., 2 to about 5, alternatively e.g., about 5 to about 50, e.g., about 5 to about 20).

For illustration, IRCs containing SEQ ID NO:17 (C869) and multiunit spacers include

5′-TCCTGGAGGGGTTGT-(C3)₁₅-T
5′-TCCTGGAGGGGTTGT-(glycerol)₁₅-T
5′-TCCTGGAGGGGTTGT-(TEG)₈-T
5′-TCCTGGAGGGGTTGT-(HEG)₄-T

where (C3)₁₅ means 15 propyl linkers connected via phosphorothioate esters; (glycerol)₁₅ means 15 glycerol linkers connected via phosphorothioate esters; (TEG)₈ means 8 triethyleneglycol linkers connected via phosphorothioate esters; and (HEG)₄ means 4 hexaethyleneglycol linkers connected via phosphorothioate esters. It will be appreciated that certain multiunit spacers have a net negative charge, and that the negative charge can be increased by increasing the number of e.g., ester-linked monomeric units.

In certain embodiments, a spacer moiety is a multivalent non-nucleic acid spacer moiety (i.e., a “multivalent spacer”). As used in this context, an IRC containing a multivalent spacer contains a spacer covalently bound to three (3) or more nucleic acid moieties. Multivalent spacers are sometimes referred to in the art as “platform molecules.” Multivalent spacers can be polymeric or nonpolymeric. Examples of suitable molecules include glycerol or substituted glycerol (e.g., 2-hydroxymethyl glycerol, levulinyl-glycerol); tetraminobenzene, heptaminobetacyclodextrin, 1,3,5-trihydroxycyclohexane, pentaerythritol and derivatives of pentaerythritol, tetraminopentaerythritol, 1,4,8,11-tetraazacyclo tetradecane (Cyclam), 1,4,7,10-tetraazacyclododecane (Cyclen), polyethyleneimine, 1,3-diamino-2-propanol and substituted derivatives, propyloxymethyl]ethyl compounds (e.g., “trebler”), polyethylene glycol derivatives such as so-called “Star PEGs” and “bPEG” (see, e.g., Gnanou et al. (1988) Makromol. Chem. 189:2885; Rein et al. (1993) Acta Polymer 44:225; U.S. Pat. No. 5,171,264), and dendrimers.

Dendrimers are known in the art and are chemically defined globular molecules, generally prepared by stepwise or reiterative reaction of multifunctional monomers to obtain a branched structure (see, e.g., Tomalia et al. (1990) Angew. Chem. Int. Ed. Engl. 29:138-75). A variety of dendrimers are known, e.g., amine-terminated polyamidoamine, polyethyleneimine and polypropyleneimine dendrimers. Exemplary dendrimers useful in the present invention include “dense star” polymers or “starburst” polymers such as those described in U.S. Pat. Nos. 4,587,329; 5,338,532; and 6,177,414, including so-called “poly(amidoamine) (“PAMAM”) dendrimers.” Still other multimeric spacer molecules suitable for use within the present invention include chemically-defined, non-polymeric valency platform molecules such as those disclosed in U.S. Pat. No. 5,552,391; and PCT application publications WO 00/75105, WO 96/40197, WO 97/46251, WO 95/07073, and WO 00/34231. Many other suitable multivalent spacers can be used and will be known to those of skill in the art.

Conjugation of a nucleic acid moiety to a platform molecule can be effected in any number of ways, typically involving one or more crosslinking agents and functional groups on the nucleic acid moiety and platform molecule. Linking groups are added to platforms using standard synthetic chemistry techniques. Linking groups can be added to nucleic acid moieties using standard synthetic techniques.

Multivalent spacers with a variety of valencies are useful in the practice of the invention, and in various embodiments the multivalent spacer of an IRC is bound to between about 3 and about 400 nucleic acid moieties, often from 3 to 100, sometimes from 3-50, frequently from 3-10, and sometimes more than 400 nucleic acid moieties. In various embodiments, the multivalent spacer is conjugated to more than 10, more than 25, more than 50, or more than 500 nucleic acid moieties (which may be the same or different). It will be appreciated that, in certain embodiments in which an IRC comprises a multivalent spacer, the invention provides a population of IRCs with slightly different molecular structures. For example, when an IRC is prepared using a dendrimer as a high valency the multivalent spacer, a somewhat heterogeneous mixture of molecules is produced, i.e., comprising different numbers (within or predominantly within a determinable range) of nucleic acid moieties joined to each dendrimer molecule.

Polysaccharides derivatized to allow linking to nucleic acid moieties can be used as spacers in IRCs. Suitable polysaccharides include naturally occurring polysaccharides (e.g., dextran) and synthetic polysaccharides (e.g., ficoll). For instance, aminoethylcarboxymethyl-ficoll (AECM-Ficoll) can be prepared by the method of Inman (1975) J. Imm. 114:704-709. AECM-Ficoll can then be reacted with a heterobifunctional crosslinking reagent, such as 6-maleimido caproic acyl N-hydroxysuccinimide ester, and then conjugated to a thiol-derivatized nucleic acid moiety (see Lee et al. (1980) Mol. Imm. 17:749-56). Other polysaccharides may be modified similarly.

It will be well within the ability of one of skill, guided by this specification and knowledge in the art, to prepare IRCs using routine methods. Techniques for making nucleic acid moieties (e.g., oligonucleotides and modified oligonucleotides) are known and described herein.

For instance, DNA or RNA polynucleotides (nucleic acid moieties) containing phosphodiester linkages are generally synthesized by repetitive iterations of the following steps: a) removal of the protecting group from the 5′-hydroxyl group of the 3′-solid support-bound nucleoside or nucleic acid, b) coupling of the activated nucleoside phosphoramidite to the 5′-hydroxyl group, c) oxidation of the phosphite triester to the phosphate triester, and d) capping of unreacted 5′-hydroxyl groups. DNA or RNA containing phosphorothioate linkages is prepared as described above, except that the oxidation step is replaced with a sulfurization step. Once the desired oligonucleotide sequence has been synthesized, the oligonucleotide is removed from the support, the phosphate triester groups are deprotected to phosphate diesters and the nucleoside bases are deprotected using aqueous ammonia or other bases. See, for example, Beaucage (1993) “Oligodeoxyribonucleotide Synthesis” in PROTOCOLS FOR OLIGONUCLEOTIDES AND ANALOGS, SYNTHESIS AND PROPERTIES (Agrawal, ed.) Humana Press, Totowa, N.J.; Warner et al. (1984) DNA 3:401; Tang et al. (2000) Org. Process Res. Dev. 4:194-198; Wyrzykiewica et al. (1994) Bioorg. & Med. Chem. Lett. 4:1519-1522; Radhakrishna et al. (1989) J. Org. Chem. 55:4693-4699. and U.S. Pat. No. 4,458,066. Programmable machines that automatically synthesize nucleic acid moieties of specified sequences are widely available. Examples include the Expedite 8909 automated DNA synthesizer (Perseptive Biosystem, Framington Mass.); the ABI 394 (Applied Biosystems, Inc., Foster City, Calif.); and the OligoPilot II (Amersham Pharmacia Biotech, Piscataway, N.J.).

Polynucleotides can be assembled in the 3′ to 5′ direction, e.g., using base-protected nucleosides (monomers) containing an acid-labile 5′-protecting group and a 3′-phosphoramidite. Examples of such monomers include 5′-O-(4,4′-dimethoxytrityl)-protected nucleoside-3′-O—(N,N-diisopropylamino) 2-cyanoethyl phosphoramidite, where examples of the protected nucleosides include, but are not limited to, N6-benzoyladenosine, N4-benzoylcytidine, N2-isobutryrylguanosine, thymidine, and uridine. In this case, the solid support used contains a 3′-linked protected nucleoside. Alternatively, polynucleotides can be assembled in the 5′ to 3′ direction using base-protected nucleosides containing an acid-labile 3′-protecting group and a 5′-phosphoramidite. Examples of such monomers include 3′-O-(4,4′-dimethoxytrityl)-protected nucleoside-5′-O—(N,N-diisopropylamino) 2-cyanoethyl phosphoramidite, where examples of the protected nucleosides include, but are not limited to, N6-benzoyladenosine, N4-benzoylcytidine, N2-isobutryrylguanosine, thymidine, and uridine (Glen Research, Sterling, Va.). In this case, the solid support used contains a 5′-linked protected nucleoside. Circular nucleic acid components can be isolated, synthesized through recombinant methods, or chemically synthesized. Chemical synthesis can be performed using any method described in the literature. See, for instance, Gao et al. (1995) Nucleic Acids Res. 23:2025-2029 and Wang et al. (1994) Nucleic Acids Res. 22:2326-2333.

Addition of non-nucleic acid spacer moieties can be accomplished using routine methods. Methods for addition of particular spacer moieties are known in the art and, for example, are described in the references cited supra. See, e.g., Durand et al. (1990) Nucleic Acids Res. 18:6353-6359. The covalent linkage between a spacer moiety and nucleic acid moiety can be any of a number of types, including phosphodiester, phosphorothioate, amide, ester, ether, thioether, disulfide, phosphoramidate, phosphotriester, phosphorodithioate, methyl phosphonate and other linkages. It will often be convenient to combine a spacer moiety(s) and a nucleic acid moiety(s) using the same phosphoramidite-type chemistry used for synthesis of the nucleic acid moiety. For example, IRCs of the invention can be conveniently synthesized using an automated DNA synthesizer (e.g., Expedite 8909; Perseptive Biosystems, Framington, Mass.) using phosphoramidite chemistry (see, e.g., Beaucage, 1993, supra; Current Protocols in Nucleic Acid Chemistry, supra). However, one of skill will understand that the same (or equivalent) synthesis steps carried out by an automated DNA synthesizer can also be carried out manually, if desired. In such a synthesis, typically, one end of the spacer (or spacer subunit for multimeric spacers) is protected with a 4,4′-dimethyoxytrityl group, while the other end contains a phosphoramidite group.

A variety of spacers with the requisite protecting and reacting groups are commercially available, for example:
triethylene glycol spacer or “TEG spacer” 9-O-(4,4′-dim ethoxytrityl)triethyleneglycol-1-O-[(2-cyanoethyl) N,N-diisopropylphosphoramidite] (Glen Research, 22825 Davis Drive, Sterling, Va.)
hexaethylene glycol spacer or “HEG spacer” 18-O-(4,4′-dimethoxytrityl)hexaethyleneglycol-1-O-[(2-cyanoethyl) N,N-diisopropylphosphoramidite] (Glen Research, Sterling, Va.)
propyl spacer 3-(4,4′-dimethoxytrityloxy)propyloxy-1-O-[(2-cyanoethyl) N,N-diisopropylphosphoramidite] (Glen Research, Sterling, Va.);
butyl spacer 4-(4,4′-dimethoxytrityloxy)butyloxy-1-O-[(2-cyanoethyl) N,N-diisopropylphosphoramidite] (Chem Genes Corporation, Ashland Technology Center, 200 Horner Ave, Ashland, Mass.)
Hexyl spacer 6-(4,4′-dimethoxytrityloxy)hexyloxy-1-O-[(2-cyanoethyl) N,N-diisopropylphosphoramidite]
2-(hydroxymethyl)ethyl spacer or “HME spacer” 1-(4,4′-dimethoxytrityloxy)-3-(levulinyloxy)-propyloxy-2-O— [(2-cyanoethyl) N,N-diisopropylphosphoramidite]; also called “asymmetrical branched” spacer
“abasic nucleotide spacer” or “abasic spacer” 5-O-(4,4′-dimethoxytrityl)-1,2-dideoxyribose-3-O-[(2-cyanoethyl) N,N-diisopropylphosphoramidite] (Glen Research, Sterling, Va.)
“symmetrical branched spacer” or “glycerol spacer” 1,3-O,O-bis(4,4′-dimethoxytritypglycerol-2-O-[(2-cyanoethyl) N,N-diisopropylphosphoramidite] (Chem Genes, Ashland, Mass.)
“trebler spacer” 2,2,2-O,O,O-tris[3-O-(4,4′-dimethoxytrityloxy)propyloxymethyl]ethyl-1-O-[(2-cyanoethyl) N,N-diisopropylphosphoramidite] (Glen Research, Sterling, Va.)
“symmetrical doubler spacer” 1,3-O,O-bis[5-O-(4,4′-dimethoxytrityloxy)pentylamido]propyl-2-O-[(2-cyanoethyl) N,N-diisopropylphosphoramidite] (Glen Research, Sterling, Va.)
“dodecyl spacer” 12-(4,4′-dimethoxytrityloxy)dodecyloxy-1-O-[(2-cyanoethyl) N,N-diisopropylphosphoramidite] (Glen Research, Sterling, Va.)

These and a large variety of other protected spacer moiety precursors (e.g., comprising DMT and phosphoramidite group protecting groups) can be purchased or can be synthesized using routine methods for use in preparing IRCs disclosed herein. The instrument is programmed according to the manufacturer's instructions to add nucleotide monomers and spacers in the desired order.

Although use of phosphoramidite chemistry is convenient for the preparation of certain IRCs, it will be appreciated that the IRCs of the invention are not limited to compounds prepared by any particular method of synthesis or preparation.

In one embodiment, IRCs with multivalent spacers conjugated to more than one type of nucleic acid moiety are prepared. For instance, platforms containing two maleimide groups (which can react with thiol-containing polynucleotides), and two activated ester groups (which can react with amino-containing nucleic acids) have been described (see, e.g., PCT application publication WO 95/07073). These two activated groups can be reacted independently of each other. This would result in an IRC containing a total of 4 nucleic acid moieties, two of each sequence.

IRCs with multivalent spacers containing two different nucleic acid sequences can also be prepared using the symmetrical branched spacer, described above, and conventional phosphoramidite chemistry (e.g., using manual or automated methods). The symmetrical branched spacer contains a phosphoramidite group and two protecting groups that are the same and are removed simultaneously. In one approach, for example, a first nucleic acid is synthesized and coupled to the symmetrical branched spacer, the protecting groups are removed from the spacer. Then two additional nucleic acids (of the same sequence) are synthesized on the spacer (using double the amount of reagents used for synthesis of a single nucleic acid moiety in each step).

A similar method can be used to connect three different nucleic acid moieties (referred to below as Nucleic acids I, II, and III) to a multivalent platform (e.g., asymmetrical branched spacer). This is most conveniently carried out using an automated DNA synthesizer. In one embodiment, the asymmetrical branched spacer contains a phosphoramidite group and two orthogonal protecting groups that can be removed independently. First, nucleic acid I is synthesized, then the asymmetrical branched spacer is coupled to nucleic acid I, then nucleic acid II is added after the selective removal of one of the protecting groups. Nucleic acid II is deprotected, and capped, and then the other protecting group on the spacer is removed. Finally, nucleic acid III is synthesized.

In some embodiments, a nucleic acid moiety(s) is synthesized, and a reactive linking group (e.g., amino, carboxylate, thio, disulfide, and the like) is added using standard synthetic chemistry techniques. The reactive linking group (which is considered to form a portion of the resulting spacer moiety) is conjugated to additional non-nucleic acid compounds to form the spacer moiety. Linking groups are added to nucleic acids using standard methods for nucleic acid synthesis, employing a variety of reagents described in the literature or commercially available. Examples include reagents that contain a protected amino group, carboxylate group, thiol group, or disulfide group and a phosphoramidite group. Once these compounds are incorporated into the nucleic acids, via the activated phosphoramidite group, and are deprotected, they provide nucleic acids with amino, carboxylate, or thiol reactivity.

Hydrophilic linkers of variable lengths are useful, for example to link nucleic acids moieties and platform molecules. A variety of suitable linkers are known. Suitable linkers include, without limitation, linear oligomers or polymers of ethylene glycol. Such linkers include linkers with the formula R¹S(CH₂CH₂O)ₙCH₂CH₂—O—(CH₂)ₘCO₂R² wherein n=0-200, m=1 or 2, R¹═H or a protecting group such as trityl, R²═H or alkyl or aryl, e.g., 4-nitrophenyl ester. These linkers are useful in connecting a molecule containing a thiol reactive group such as haloaceyl, maleiamide, etc., via a thioether to a second molecule which contains an amino group via an amide bond. The order of attachment can vary, i.e., the thioether bond can be formed before or after the amide bond is formed. Other useful linkers include Sulfo-SMCC (sulfosuccinimidyl 4-[N-maleimidomethyl]-cyclohexane-1-carboxylate) Pierce Chemical Co. product 22322; Sulfo-EMCS (N-[ε-maleimidocaproyloxyl sulfosuccinimide ester) Pierce Chemical Co. product 22307; Sulfo-GMBS (N-[γ-maleimidobutyryloxy]sulfosuccinimide ester) Pierce Chemical Co. product 22324 (Pierce Chemical Co., Rockford, Ill.), and similar compounds of the general formula maleimido-R—C(O)NHS ester, where R=alkyl, cyclic alkyl, polymers of ethylene glycol, and the like.

Particularly useful methods for covalently joining nucleic acid moieties to multivalent spacers are described in the references cited supra.

In certain embodiments, a polypeptide is used as a multivalent spacer moiety to which a plurality of nucleic acid moieties are covalently conjugated, directly or via linkers, to form a “proteinaceous IRC.” The polypeptide can be a carrier (e.g., albumin). Typically, a proteinaceous IRC comprises at least one, and usually several or many nucleic acid moieties that (a) are between 2 and 7, more often between 4 and 7 nucleotides in length, alternatively between 2 and 6, 2 and 5, 4 and 6, or 4 and 5 nucleotides in length and/or (b) have inferior isolated immunomodulatory activity or do not have isolated immunomodulatory activity. Methods of making a proteinaceous IRC will be apparent to one of skill upon review of the present disclosure. A nucleic acid, for example, can be covalently conjugated to a polypeptide spacer moiety by art known methods including linkages between a 3′ or 5′ end of a nucleic acid moiety (or at a suitably modified base at an internal position in the a nucleic acid moiety) and a polypeptide with a suitable reactive group (e.g., an N-hydroxysuccinimide ester, which can be reacted directly with the N⁴ amino group of cytosine residues). As a further example, a polypeptide can be attached to a free 5′-end of a nucleic acid moiety through an amine, thiol, or carboxyl group that has been incorporated into nucleic acid moiety. Alternatively, the polypeptide can be conjugated to a spacer moiety, as described herein. Further, a linking group comprising a protected amine, thiol, or carboxyl at one end, and a phosphoramidite can be covalently attached to a hydroxyl group of a polynucleotide, and, subsequent to deprotection, the functionality can be used to covalently attach the IRC to a peptide.

Antigen

The NISCs of the invention may include any antigen involved in an unwanted or inapproriate immune reaction or response. Also, the NISCs may include any antigen to which an individual may be at risk of developing an unwanted or inapproriate immune reaction or response.

In some embodiments, the antigen is an autoantigen. Autoantigens are known for a number of autoimmune diseases. For example, Grave's disease is characterized by production of autoantibodies to the thyroid-stimulating hormone receptor of the thyroid gland, Hashimoto's thyroiditis by autoantibodies and T cells to thyroid antigens (e.g., thyroid peroxidase), and type I diabetes by T cells and autoantibodies to β cell antigens (e.g., glutamic acid decarboxylase and insulin).

Other examples of autoantigens involved in autoimmune diseases include, but are not limited to, cytochrome P450 antigens in Addison's disease, myelin proteins (e.g., myelin basic protein) in MS, uveal antigens in uveitis, gastric parietal cell antigens (e.g., H⁺/ATPase, intrinsic factor) in pernicious anemia, transglutaminase in gluten enteropathy, myocardial cell proteins (e.g., myosin) in myocarditis and rheumatic heart disease, platelet antigens (e.g., GP IIb/IIIa) in idiopathic thrombocytopenic purpura, red blood cell membrane proteins in autoimmune hemolytic anemia, neutrophil membrane proteins in autoimmune neutropenia, basement membrane antigens (e.g., type IV collagen α3 chain) in Goodpasture's disease, intrahepatic bile duct/mitochondrial antigens (e.g., 2-oxoacid dehydrogenase complexes) for primary biliary cirrhosis, hepatocyte antigens (e.g., cytochrome P450, 206) for autoimmune hepatitis, acetylcholine receptors for myasthenia gravis, and desmogleins for pemphigus and other bullous diseases.

In some embodiments, the antigen is an alloantigen. Alloantigens are generally cellular antigens that vary in structure among individual members of a single species. Alloantigens from one individual can be recognized as foreign antigens by other members of the same species and are often the basis for graft rejection reactions. Examples of alloantigens include, but are not limited to major histocompatability complex (MHC) class I and class II antigens, minor histocompatability antigens, certain tissue-specific antigens, endothelial glycoproteins such as blood group antigens, and carbohydrate determinants.

In some embodiments, the antigen is an allergen. Examples of allergens are provided in Table 1. Preparation of many allergens is well-known in the art, including, but not limited to, preparation of ragweed pollen allergen Antigen E (Amb a I) (Rafnar et al. (1991) J. Biol. Chem. 266:1229-1236), grass allergen Lol p 1 (Tamborini et al. (1997) Eur. J. Biochem. 249:886-894), major dust mite allergens Der pI and Der PII (Chua et al. (1988) J. Exp. Med. 167:175-182; Chua et al. (1990) Int. Arch. Allergy Appl. Immunol. 91:124-129), domestic cat allergen Fel d I (Rogers et al. (1993) Mol. Immunol. 30:559-568), white birch pollen Bet v1 (Breiteneder et al. (1989) EMBO J. 8:1935-1938), Japanese cedar allergens Cry j 1 and Cry j 2 (Kingetsu et al. (2000) Immunology 99:625-629), and protein antigens from other tree pollen (Elsayed et al. (1991) Scand. J. Clin. Lab. Invest. Suppl. 204:17-31). As indicated, allergens from trees are known, including allergens from birch, juniper and Japanese cedar. Preparation of protein antigens from grass pollen for in vivo administration has been reported.

In some embodiments, the allergen is a food allergen, including, but not limited to, peanut allergen, for example Ara h I (Stanley et al. (1996) Adv. Exp. Med. Biol. 409:213-216) or Ara h II; walnut allergen, for example, Jug r I (Tueber et al. (1998) J. Allergy Clin. Immunol. 101:807-814); brazil nut allergen, for example, albumin (Pastorello et al. (1998) J. Allergy Clin. Immunol. 102:1021-1027; shrimp allergen, for example, Pen a I (Reese et al. (1997) Int. Arch. Allergy Immunol. 113:240-242); egg allergen, for example, ovomucoid (Crooke et al. (1997) J. Immunol. 159:2026-2032); milk allergen, for example, bovine β-lactoglobin (Selot al. (1999) Clin. Exp. Allergy 29:1055-1063); fish allergen, for example, parvalbumins (Van Do et al. (1999) Scand. J. Immunol. 50:619-625; Galland et al. (1998) J. Chromatogr. B. Biomed. Sci. Appl. 706:63-71). In some embodiments, the allergen is a latex allergen, including but not limited to, Hey b 7 (Sowka et al. (1998) Eur. J. Biochem. 255:213-219). Table 1 shows an exemplary list of allergens that may be used.

TABLE 1
ALLERGENS

| Group | Allergen | Reference |
|---|---|---|
| ANIMALS: | | |
| CRUSTACEA | | |
| Shrimp/lobster | tropomyosin | Leung et al. (1996) J. Allergy Clin. Immunol. 98: 954-961 |
| | Pan s I | Leung et al. (1998) Mol. Mar. Biol. Biotechnol. 7: 12-20 |
| INSECTS | | |
| Ant | Sol i 2 (venom) | Schmidt et al. J Allergy Clin Immunol., 1996, 98: 82-8 |
| Bee | Phospholipase A2 (PLA) | Muller et al. J Allergy Clin Immunol, 1995, 96: 395-402 |
| | | Forster et al. J Allergy Clin Immunol, 1995, 95: 1229-35 |
| | | Muller et al. Clin Exp Allergy, 1997, 27: 915-20 |
| | Hyaluronidase (Hya) | Soldatova et al. J Allergy Clin Immunol, 1998, 101: 691-8 |
| Cockroach | Bla g Bd9OK | Helm et al. J Allergy Clin Immunol, 1996, 98: 172-180 |
| | Bla g 4 (a calycin) | Vailes et al. J Allergy Clin Immunol, 1998, 101: 274-280 |
| | Glutathione | Arruda et al. J Biol Chem, 1997, 272: 20907-12 |
| | S-transferase | |
| | Per a 3 | Wu et al. Mol Immunol, 1997, 34: 1-8 |
| Dust mite | Der p 2 (major allergen) | Lynch et al. J Allergy Clin Immunol, 1998, 101: 562-4 |
| | | Hakkaart et al. Clin Exp Allergy, 1998, 28: 169-74 |
| | | Hakkaart et al. Clin Exp Allergy, 1998, 28: 45-52 |
| | | Hakkaart et al. Int Arch Allergy Immunol, 1998, 115 |
| | | (2): 150-6 |
| | | Mueller et al. J Biol Chem, 1997, 272: 26893-8 |
| | Der p2 variant | Smith et al. J Allergy Clin Immunol, 1998, 101: 423-5 |
| | Der f2 | Yasue et al. Clin Exp Immunol, 1998, 113: 1-9 |
| | | Yasue et al. Cell Immunol, 1997, 181: 30-7 |
| | Der p10 | Asturias et al. Biochim Biophys Acta, 1998, 1397: 27-30 |
| | Tyr p 2 | Eriksson et al. Eur J Biochem, 1998 |
| Hornet | Antigen 5 aka Dol m V | Tomalski et al. Arch Insect Biochem Physiol, 1993, |
| | (venom) | 22: 303-13 |
| Mosquito | Aed a I (salivary | Xu et al. Int Arch Allergy Immunol, 1998, 115: 245-51 |
| | apyrase) | |
| Yellow jacket | antigen 5, hyaluronidase | King et al. J Allergy Clin Immunol, 1996, 98: 588-600 |
| | and phospholipase | |
| | (venom) | |
| MAMMALS | | |
| Cat | Fel d I | Slunt et al. J Allergy Clin Immunol, 1995, 95: 1221-8 |
| | | Hoffmann et al. (1997) J Allergy Clin Immunol 99: 227-32 |
| | | Hedlin Curr Opin Pediatr, 1995, 7: 676-82 |
| Cow | Bos d 2 (dander; a | Zeiler et al. J Allergy Clin Immunol, 1997, 100: 721-7 |
| | lipocalin) | Rautiainen et al. Biochem Bioph. Res Comm., 1998, |
| | | 247: 746-50 |
| | β-lactoglobulin (BLG, | Chatel et al. Mol Immunol, 1996, 33: 1113-8 |
| | major cow milk allergen) | Lehrer et al. Crit Rev Food Sci Nutr, 1996, 36: 553-64 |
| Dog | Can f I and Can f 2, | Konieczny et al. Immunology, 1997, 92: 577-86 |
| | salivary lipocalins | Spitzauer et al. J Allergy Clin Immunol, 1994, 93: 614-27 |
| | | Vrtala et al. J Immunol, 1998, 160: 6137-44 |
| Horse | Equ c1 (major allergen, | Gregoire et al. J Biol Chem, 1996, 271: 32951-9 |
| | a lipocalin) | |
| Mouse | mouse urinary protein | Konieczny et al. Immunology, 1997, 92: 577-86 |
| | (MUP) | |
| OTHER | | |
| MAMMALIAN | | |
| ALLERGENS | | |
| Insulin | | Ganz et al. J Allergy Clin Immunol, 1990, 86: 45-51 |
| | | Grammer et al. J Lab Clin Med, 1987, 109: 141-6 |
| | | Gonzalo et al. Allergy, 1998, 53: 106-7 |
| Interferons | interferon alpha 2c | Detmar et al. Contact Dermatis, 1989, 20: 149-50 |
| MOLLUSCS | topomyosin | Leung et al. J Allergy Clin Immunol, 1996, 98: 954-61 |
| PLANT | | |
| ALLERGENS: | | |
| Barley | Hor v 9 | Astwood et al. Adv Exp Med Biol, 1996, 409: 269-77 |
| Birch | pollen allergen, Bet v 4 | Twardosz et al. Biochem Bioph. Res Comm., 1997, 239: 197 |
| | rBet v 1 Bet v 2: | Pauli et al. J Allergy Clin Immunol, 1996, 97: 1100-9 |
| | (profilin) | van Neerven et al. Clin Exp Allergy, 1998, 28: 423-33 |
| | | Jahn-Schmid et al. Immunotechnology, 1996, 2: 103-13 |
| | | Breitwieser et al. Biotechniques, 1996, 21: 918-25 |
| | | Fuchs et al. J Allergy Clin Immunol, 1997, 100: 3 56-64 |
| Brazil nut | globulin | Bartolome et al. Allergol Immunopathol, 1997, 25: 135-44 |
| Cherry | Pru a I (major allergen) | Scheurer et al. Mol Immunol, 1997, 34: 619-29 |
| Corn | Zml3 (pollen) | Heiss et al. FEBS Lett, 1996, 381: 217-21 |
| | | Lehrer et al. Int Arch Allergy Immunol, 1997, 113: 122-4 |
| Grass | Phl p 1, Phl p 2, Phl p 5 | Bufe et al. Am J Respir Crit Care Med, 1998, 157: 1269-76 |
| | (timothy grass pollen) | Vrtala et al. J Immunol Jun. 15, 1998, 160: 6137-44 |
| | | Niederberger et al. J Allergy Clin Immun., 1998, 101: 258 |
| | | Schramm et al. Eur J Biochem, 1998, 252: 200-6 |
| | Hol 1 5 velvet grass | Zhang et al. J Immunol, 1993, 151: 791-9 |
| | pollen | |
| | Bluegrass allergen | Smith et al. Int Arch Allergy Immunol, 1997, 114: 265-71 |
| | Cyn d 7 Bermuda grass | Asturias et al. Clin Exp Allergy, 1997, 27: 1307-13 |
| | Cyn d 12 (a profilin) | Fuchs et al. J Allergy Clin Immunol, 1997, 100: 356-64 |
| Japanese Cedar | Jun a 2 (Juniperus ashei) | Yokoyama et al. Biochem. Biophys. Res. Commun., 2000, |
| | | 275: 195-202 |
| | Cry j 1, Cry j 2 | Kingetsu et al. Immunology, 2000, 99: 625-629 |
| | (Cryptomeria japonica) | |
| Juniper | Jun o 2 (pollen) | Tinghino et al. J Allergy Clin Immunol, 1998, 101: 772-7 |
| Latex | Hev b 7 | Sowka et al. Eur J Biochem, 1998, 255: 213-9 |
| | | Fuchs et al. J Allergy Clin Immunol, 1997, 100: 3 56-64 |
| Mercurialis | Mer a I (profilin) | Vallverdu et al. J Allergy Clin Immunol, 1998, 101: 3 63 |
| Mustard | Sin a I (seed) | Gonzalez de la Pena et al. Biochem Bioph. Res Comm., |
| (Yellow) | | 1993, 190: 648-53 |
| Oilseed rape | Bra r I pollen allergen | Smith et al. Int Arch Allergy Immunol, 1997, 114: 265-71 |
| Peanut | Ara h I | Stanley et al. Adv Exp Med Biol, 1996, 409: 213-6 |
| | | Burks et al. J Clin Invest, 1995, 96: 1715-21 |
| | | Burks et al. Int Arch Allergy Immunol, 1995, 107: 248-50 |
| Poa pratensis | Poa p9 | Parronchi et al. Eur J Immunol, 1996, 26: 697-703 |
| | | Astwood et al. Adv Exp Med Biol, 1996, 409: 269-77 |
| Ragweed | Amb a I | Sun et al. Biotechnology August 1995, 13: 779-86 |
| | | Hirschwehr et al. J Allergy Clin Immunol, 1998, 101: 196 |
| | | Casale et al. J Allergy Clin Immunol, 1997, 100: 110-21 |
| Rye | Lol p I | Tamborini et al. Eur J Biochem, 1997, 249: 886-94 |
| Walnut | Jug r I | Teuber et al. J Allergy Clin Immun., 1998, 101: 807-14 |
| Wheat | allergen | Fuchs et al. J Allergy Clin Immunol, 1997, 100: 356-64 |
| | | Donovan et al. Electrophoresis, 1993, 14: 917-22 |
| FUNGI: | | |
| Aspergillus | Asp f 1, Asp f 2, Asp f3, | Crameri et al. Mycoses, 1998, 41 Suppl 1: 56-60 |
| | Asp f 4, rAsp f 6 | Hemmann et al. Eur J Immunol, 1998, 28: 1155-60 |
| | | Banerjee et al. J Allergy Clin Immunol, 1997, 99: 821-7 |
| | | Crameri Int Arch Allergy Immunol, 1998, 115: 99-114 |
| | | Crameri et al. Adv Exp Med Biol, 1996, 409: 111-6 |
| | | Moser et al. J Allergy Clin Immunol, 1994, 93: 1-11 |
| | Manganese superoxide | Mayer et al. Int Arch Allergy Immunol, 1997, 113: 213-5 |
| | dismutase (MNSOD) | |
| Blomia | allergen | Caraballo et al. Adv Exp Med Biol, 1996, 409: 81-3 |
| Penicillinium | allergen | Shen et al. Clin Exp Allergy, 1997, 27: 682-90 |
| Psilocybe | Psi c 2 | Horner et al. Int Arch Allergy Immunol, 1995, 107: 298 |

Gliadin is the antigen in wheat gluten that is the source of celiac disease. Thus, in some embodiments, the antigen is a wheat gluten antigen, such as gliadin.

In some embodiments, the antigen can be from an infectious agent, including protozoan, bacterial, fungal (including unicellular and multicellular), and viral infectious agents. For example, antigens from parasitic organisms include schistosome egg antigens (e.g., Sm-p40) from Schistosome species (e.g., S. mansoni) and antigens from Toxoplasma species (e.g., T. gondii). See, for example, Stadecker et al. (1998) Parasite Immunol. 20:217-221; Subauste et al. (1993) Curr. Opin. Immunol. 5:532-527. In such cases, the infectious agent antigen is one to which an unwanted immune response has occurred or is at risk of occurring.

Antigens may be isolated from their source using purification techniques known in the art or, more conveniently, may be produced using recombinant methods.

Antigenic peptides can include purified native peptides, synthetic peptides, recombinant proteins, crude protein extracts, attenuated or inactivated viruses, cells, micro-organisms, or fragments of such peptides. Immunomodulatory peptides can be native or synthesized chemically or enzymatically. Any method of chemical synthesis known in the art is suitable. Solution phase peptide synthesis can be used to construct peptides of moderate size or, for the chemical construction of peptides, solid phase synthesis can be employed. Atherton et al. (1981) Hoppe Seylers Z. Physiol. Chem. 362:833-839. Proteolytic enzymes can also be utilized to couple amino acids to produce peptides. Alternatively, the peptide can be obtained by using the biochemical machinery of a cell, or by isolation from a biological source. Recombinant DNA techniques can be employed for the production of peptides. Peptides can also be isolated using standard techniques such as affinity chromatography.

Generally, the antigens are peptides, lipids (e.g., sterols excluding cholesterol, fatty acids, and phospholipids), polysaccharides, gangliosides and glycoproteins. These can be obtained through several methods known in the art, including isolation and synthesis using chemical and enzymatic methods. In certain cases, such as for many sterols, fatty acids and phospholipids, the antigenic portions of the molecules are commercially available.

Antigens derived from infectious agents may be obtained using methods known in the art, for example, from native viral or bacterial extracts, from cells infected with the infectious agent, from purified polypeptides, from recombinantly produced polypeptides and/or as synthetic peptides.

NISC Formation

In NISCs of the invention, the non-immunostimulatory polynucleotide may be coupled with the antigen in a number of ways, including conjugation (linkage), encapsulation, via affixation to a platform or adsorption onto a surface. The polynucleotide portion can be coupled with the antigen portion of a conjugate involving covalent and/or non-covalent interactions. Generally, a non-immunostimulatory polynucleotide and antigen are linked in a manner that allows enhanced or facilitated uptake of the antigen by DCs and/or APCs with little or no DC and/or APC activation or maturation. Alternatively, a non-immunostimulatory polynucleotide and antigen are linked in a manner that allows increased antigen presentation by DCs and/or APCs with little or no DC and/or APC activation or maturation.

The link between the portions can be made at the 3′ or 5′ end of the non-immunostimulatory polynucleotide, or at a suitably modified base at an internal position in the polynucleotide. If the antigen is a peptide and contains a suitable reactive group (e.g., an N-hydroxysuccinimide ester) it can be reacted directly with the N⁴ amino group of cytosine residues. Depending on the number and location of cytosine residues in the polynucleotide, specific coupling at one or more residues can be achieved.

Alternatively, modified oligonucleosides, such as are known in the art, can be incorporated at either terminus, or at internal positions in the non-immunostimulatory polynucleotide. These can contain blocked functional groups which, when deblocked, are reactive with a variety of functional groups which can be present on, or attached to, the antigen of interest.

Where the antigen is a peptide or polypeptide, this portion of the conjugate can be attached to the 3′-end of the polynucleotide through solid support chemistry. For example, the polynucleotide portion can be added to a polypeptide portion that has been pre-synthesized on a support. Alternatively, the non-immunostimulatory polynucleotide can be synthesized such that it is connected to a solid support through a cleavable linker extending from the 3′-end. Upon chemical cleavage of the polynucleotide from the support, a terminal thiol group is left at the 3′-end of the oligonucleotide or a terminal amino group is left at the 3′-end of the oligonucleotide. Conjugation of the amino-modified non-immunostimulatory polynucleotide to amino groups of the peptide can be performed as described in Benoit et al. (1987) Neuromethods 6:43-72. Conjugation of the thiol-modified non-immunostimulatory polynucleotide to carboxyl groups of the peptide can be performed as known in the art. Coupling of a polynucleotide carrying an appended maleimide to the thiol side chain of a cysteine residue of a peptide is also known in the art. See, for example, Haralambidis et al. (1990a) Nucleic Acids Res. 18:493-499; Haralambidis et al. (1990b) Nucleic Acids Res. 18:501-505; Zuckermann et al. (1987) Nucleic Acids Res. 15:5305-5321; Corey et al. (1987) Science 238:1401-1403); Nelson et al. (1989) Nucleic Acids Res. 17:1781-1794; Tung et al. (1991) Bioconjug. Chem. 2:464-465.

The peptide or polypeptide portion of the conjugate can be attached to the 5′-end of the polynucleotide through an amine, thiol, or carboxyl group that has been incorporated into the oligonucleotide during its synthesis. Preferably, while the oligonucleotide is fixed to the solid support, a linking group comprising a protected amine, thiol, or carboxyl at one end, and a phosphoramidite at the other, is covalently attached to the 5′-hydroxyl. Agrawal et al. (1986) Nucleic Acids Res. 14:6227-6245; Connolly (1985) Nucleic Acids Res. 13:4485-4502; Kremsky et al. (1987) Nucleic Acids Res. 15:2891-2909; Connolly (1987) Nucleic Acids Res. 15:3131-3139; Bischoff et al. (1987) Anal. Biochem. 164:336-344; Blanks et al. (1988) Nucleic Acids Res. 16:10283-10299; and U.S. Pat. Nos. 4,849,513, 5,015,733, 5,118,800, and 5,118,802. Subsequent to deprotection, the amine, thiol, and carboxyl functionalities can be used to covalently attach the oligonucleotide to a peptide. Benoit et al. (1987).

A NISC can also be formed through non-covalent interactions, such as ionic bonds, hydrophobic interactions, hydrogen bonds and/or van der Waals attractions.

Non-covalently linked conjugates can include a non-covalent interaction such as a biotin-streptavidin complex. A biotinyl group can be attached, for example, to a modified base of a polynucleotide. Roget et al. (1989) Nucleic Acids Res. 17:7643-7651. Incorporation of a streptavidin moiety into the peptide portion allows formation of a non-covalently bound complex of the streptavidin conjugated peptide and the biotinylated oligonucleotide.

Non-covalent associations can also occur through ionic interactions involving a polynucleotide and residues within the antigen, such as charged amino acids, or through the use of a linker portion comprising charged residues that can interact with both the polynucleotide and the antigen. For example, non-covalent conjugation can occur between a generally negatively-charged polynucleotide and positively-charged amino acid residues of a peptide, e.g., polylysine, polyarginine and polyhistidine residues.

Non-covalent conjugation between non-immunostimulatory polynucleotide and antigens can occur through DNA binding motifs of molecules that interact with DNA as their natural ligands. For example, such DNA binding motifs can be found in transcription factors and anti-DNA antibodies.

The linkage of the non-immunostimulatory polynucleotide to a lipid can be formed using standard methods. These methods include, but are not limited to, the synthesis of oligonucleotide-phospholipid conjugates (Yanagawa et al. (1988) Nucleic Acids Symp. Ser. 19:189-92), oligonucleotide-fatty acid conjugates (Grabarek et al. (1990) Anal. Biochem. 185:131-35; and Staros et al. (1986) Anal. Biochem. 156:220-2), and oligonucleotide-sterol conjugates. Boujrad et al. (1993) Proc. Natl. Acad. Sci. USA 90:5728-31.

The linkage of the oligonucleotide to an oligosaccharide can be formed using standard known methods. These methods include, but are not limited to, the synthesis of oligonucleotide-oligosaccharide conjugates, wherein the oligosaccharide is a moiety of an immunoglobulin. O'Shannessy et al. (1985) J. Applied Biochem. 7:347-55.

The linkage of a circular non-immunostimulatory polynucleotide to an antigen can be formed in several ways. Where the circular polynucleotide is synthesized using recombinant or chemical methods, a modified nucleoside is suitable. Standard linking technology can then be used to connect the circular polynucleotide to the antigen. Goodchild (1990) Bioconjug. Chem. 1:165. Where the circular polynucleotide is isolated, or synthesized using recombinant or chemical methods, the linkage can be formed by chemically activating, or photoactivating, a reactive group (e.g. carbene, radical) that has been incorporated into the antigen.

Additional methods for the attachment of peptides and other molecules to oligonucleotides can be found in U.S. Pat. No. 5,391,723; Kessler (1992) “Nonradioactive labeling methods for nucleic acids” in Kricka (ed.) Nonisotopic DNA Probe Techniques, Academic Press; and Geoghegan et al. (1992) Bioconjug. Chem. 3:138-146.

In some embodiments, a non-immunostimulatory polynucleotide and antigen are coupled by encapsulation. In other embodiments, a non-immunostimulatory polynucleotide and antigen are coupled by linkage to a platform molecule. A “platform molecule” (also termed “platform”) is a molecule containing sites which allow for attachment of the polynucleotide and antigen(s). In other embodiments, a non-immunostimulatory polynucleotide and antigen are coupled by adsorption onto a surface, preferably a carrier particle.

In some embodiments, a non-immunostimulatory polynucleotide and antigen are coupled by encapsulation. In some instances, the composition comprising a non-immunostimulatory polynucleotide, antigen, and encapsulating agent is in the form of oil-in-water emulsions, microparticles and/or liposomes. Preferably, oil-in-water emulsions, microparticles and/or liposomes encapsulating a non-immunostimulatory polynucleotide and antigen are in the form of particles from about 0.04 μm to about 100 μm in size, preferably any of the following ranges: from about 0.1 μm to about 20 μm; from about 0.15 μm to about 10 μm; from about 0.05 μm to about 1.00 μm; from about 0.05 μm to about 0.5 μm.

Colloidal dispersion systems, such as microspheres, beads, macromolecular complexes, nanocapsules and lipid-based system, such as oil-in-water emulsions, micelles, mixed micelles and liposomes can provide effective encapsulation of non-immunostimulatory polynucleotide and antigen compositions.

The encapsulation composition may further comprise any of a wide variety of components. These include, but are not limited to, alum, lipids, phospholipids, lipid membrane structures (LMS), polyethylene glycol (PEG) and other polymers, such as polypeptides, glycopeptides, and polysaccharides.

Polypeptides suitable for encapsulation components include any known in the art and include, but are not limited to, fatty acid binding proteins. Modified polypeptides contain any of a variety of modifications, including, but not limited to glycosylation, phosphorylation, myristylation, sulfation and hydroxylation. As used herein, a suitable polypeptide is one that will protect a NISC composition to preserve integrity thereof until taken up by the DC and/or APC. Examples of binding proteins include, but are not limited to, albumins such as bovine serum albumin and pea albumin.

Other suitable polymers can be any known in the art of pharmaceuticals and include, but are not limited to, naturally-occurring polymers such as dextrans, hydroxyethyl starch, and polysaccharides, and synthetic polymers. Examples of naturally occurring polymers include proteins, glycopeptides, polysaccharides, dextran and lipids. The additional polymer can be a synthetic polymer. Examples of synthetic polymers which are suitable for use in the present invention include, but are not limited to, polyalkyl glycols (PAG) such as PEG, polyoxyethylated polyols (POP), such as polyoxyethylated glycerol (POG), polytrimethylene glycol (PTG) polypropylene glycol (PPG), polyhydroxyethyl methacrylate, polyvinyl alcohol (PVA), polyacrylic acid, polyethyloxazoline, polyacrylamide, polyvinylpyrrolidone (PVP), polyamino acids, polyurethane and polyphosphazene. The synthetic polymers can also be linear or branched, substituted or unsubstituted, homopolymeric, co-polymers, or block co-polymers of two or more different synthetic monomers.

The PEGs for use in encapsulation compositions of the present invention are either purchased from chemical suppliers or synthesized using techniques known to those of skill in the art.

The term “LMS” as used herein means lamellar lipid particles wherein polar head groups of a polar lipid are arranged to face an aqueous phase of an interface to form membrane structures. Examples of the LMSs include liposomes, micelles, cochleates (i.e., generally cylindrical liposomes), microemulsions, unilamellar vesicles, multilamellar vesicles, and the like. As used herein, a “liposome” or “lipid vesicle” is a small vesicle bounded by at least one and possibly more than one bilayer lipid membrane. Liposomes are made artificially from phospholipids, glycolipids, lipids, steroids such as cholesterol, related molecules, or a combination thereof by any technique known in the art, including but not limited to sonication, extrusion, or removal of detergent from lipid-detergent complexes. A liposome can also optionally comprise additional components, such as a tissue targeting component. It is understood that a “lipid membrane” or “lipid bilayer” need not consist exclusively of lipids, but can additionally contain any suitable other components, including, but not limited to, cholesterol and other steroids, lipid-soluble chemicals, proteins of any length, and other amphipathic molecules, providing the general structure of the membrane is a sheet of two hydrophilic surfaces sandwiching a hydrophobic core. For a general discussion of membrane structure, see The Encyclopedia of Molecular Biology by J. Kendrew (1994). For suitable lipids see e.g., Lasic (1993) “Liposomes: from Physics to Applications” Elsevier, Amsterdam.

Processes for preparing liposomes containing non-immunostimulatory polynucleotide and antigen are known in the art. The lipid vesicles can be prepared by any suitable technique known in the art including, but are not limited to, microencapsulation, microfluidization, LLC method, ethanol injection, freon injection, the “bubble” method, detergent dialysis, hydration, sonication, and reverse-phase evaporation. Reviewed in Watwe et al. (1995) Curr. Sci. 68:715-724. Techniques may be combined in order to provide vesicles with the most desirable attributes.

The LMS compositions of the present invention can additionally comprise surfactants. Surfactants can be cationic, anionic, amphiphilic, or nonionic. A preferred class of surfactants are nonionic surfactants; particularly preferred are those that are water soluble.

The invention encompasses use of LMSs containing tissue or cellular targeting components. Such targeting components are components of a LMS that enhance its accumulation at certain tissue or cellular sites in preference to other tissue or cellular sites when administered to an intact animal, organ, or cell culture. A targeting component is generally accessible from outside the liposome, and is therefore preferably either bound to the outer surface or inserted into the outer lipid bilayer. A targeting component can be inter alia a peptide, a region of a larger peptide, an antibody specific for a cell surface molecule or marker, or antigen binding fragment thereof, a nucleic acid, a carbohydrate, a region of a complex carbohydrate, a special lipid, or a small molecule such as a drug, hormone, or hapten, attached to any of the aforementioned molecules. Antibodies with specificity toward cell type-specific cell surface markers are known in the art and are readily prepared by methods known in the art.

Preferably, NISCs comprising LMSs with targeting components are targeted to any APC or DC or to any organs particularly containing APCs or DCs. Such target cells and organs include, but are not limited to, APCs, such as macrophages, dendritic cells and lymphocytes, lymphatic structures, such as lymph nodes and the spleen, and nonlymphatic structures, particularly those in which dendritic cells are found.

In embodiments in which a non-immunostimulatory polynucleotide and antigen are coupled by linkage to a platform molecule, the platform may be proteinaceous or non-proteinaceous (i.e., organic). Examples of proteinaceous platforms include, but are not limited to, albumin, gammaglobulin, immunoglobulin (IgG) and ovalbumin. Borel et al. (1990) Immunol. Methods 126:159-168; Dumas et al. (1995) Arch. Dematol. Res. 287:123-128; Borel et al. (1995) Int. Arch. Allergy Immunol. 107:264-267; Borel et al. (1996) Ann. N.Y. Acad. Sci. 778:80-87. A platform is multi-valent (i.e., contains more than one binding, or linking, site) to accommodate binding to both a non-immunostimulatory polynucleotide and antigen. Accordingly, a platform may contain 2 or more, 3 or more, 4 or more, 5 or more, 6 or more, 7 or more, 8 or more, 9 or more, 10 or more binding or linking sites Other examples of polymeric platforms are dextran, polyacrylamide, ficoli, carboxymethylcellulose, polyvinyl alcohol, and poly D-glutamic acid/D-lysine.

The principles of using platform molecules are well understood in the art. Generally, a platform contains, or is derivatized to contain, appropriate binding sites for polynucleotide and antigen. In addition, or alternatively, polynucleotide and/or antigen is derivatized to provide appropriate linkage groups. For example, a simple platform is a bi-functional linker (i.e., has two binding sites), such as a peptide. Further examples are discussed below.

Platform molecules may be biologically stabilized, i.e., they exhibit an in vivo excretion half-life often of hours to days to months to confer therapeutic efficacy, and are preferably composed of a synthetic single chain of defined composition. They generally have a molecular weight in the range of about 200 to about 1,000,000, preferably any of the following ranges: from about 200 to about 500,000; from about 200 to about 200,000; from about 200 to about 50,000 (or less, such as 30,000). Examples of valency platform molecules are polymers (or are comprised of polymers) such as polyethylene glycol (PEG; preferably having a molecular weight of about 200 to about 8000), poly-D-lysine, polyvinyl alcohol, polyvinylpyrrolidone, D-glutamic acid and D-lysine (in a ratio of 3:2). Other molecules that may be used are albumin and IgG.

Other platform molecules suitable for use within the present invention are the chemically-defined, non-polymeric valency platform molecules disclosed in U.S. Pat. No. 5,552,391. Other homogeneous chemically-defined valency platform molecules suitable for use within the present invention are derivatized 2,2′-ethylenedioxydiethylamine (EDDA) and triethylene glycol (TEG).

Additional suitable valency platform molecules include, but are not limited to, tetraminobenzene, heptaminobetacyclodextrin, tetraminopentaerythritol, 1,4,8,11-tetraazacyclotetradecane (Cyclam) and 1,4,7,10-tetraazacyclododecane (Cyclen).

In general, these platforms are made by standard chemical synthesis techniques. PEG must be derivatized and made multivalent, which is accomplished using standard techniques. Some substances suitable for conjugate synthesis, such as PEG, albumin, and IgG are available commercially.

Conjugation of a non-immunostimulatory polynucleotide and antigen to a platform molecule may be effected in any number of ways, typically involving one or more crosslinking agents and functional groups on the antigen and polynucleotide platform and platform molecule. Platforms and non-immunostimulatory polynucleotide and antigen must have appropriate linking groups. Linking groups are added to platforms using standard synthetic chemistry techniques. Linking groups may be added to polypeptide antigens and polynucleotide using either standard solid phase synthetic techniques or recombinant techniques. Recombinant approaches may require post-translational modification in order to attach a linker, and such methods are known in the art.

As an example, polypeptides contain amino acid side chain moieties containing functional groups such as amino, carboxyl or sulfhydryl groups that serve as sites for coupling the polypeptide to the platform. Residues that have such functional groups may be added to the polypeptide if the polypeptide does not already contain these groups. Such residues may be incorporated by solid phase synthesis techniques or recombinant techniques, both of which are well known in the peptide synthesis arts. When the polypeptide has a carbohydrate side chain(s) (or if the antigen is a carbohydrate), functional amino, sulfhydryl and/or aldehyde groups may be incorporated therein by conventional chemistry. For instance, primary amino groups may be incorporated by reaction of the oxidized sugar with ethylenediamine in the presence of sodium cyanoborohydride, sulfhydryls may be introduced by reaction of cysteamine dihydrochloride followed by reduction with a standard disulfide reducing agent, while aldehyde groups may be generated following periodate oxidation. In a similar fashion, the platform molecule may also be derivatized to contain functional groups if it does not already possess appropriate functional groups.

Hydrophilic linkers of variable lengths are useful for connecting non-immunostimulatory polynucleotide and antigen to platform molecules. Suitable linkers include linear oligomers or polymers of ethylene glycol. Such linkers include linkers with the formula R¹S(CH₂CH₂O)ₙCH₂CH₂—O—(CH₂)ₘCO₂R² wherein n=0-200, m=1 or 2, R¹═H or a protecting group such as trityl, R²═H or alkyl or aryl, e.g., 4-nitrophenyl ester. These linkers are useful in connecting a molecule containing a thiol reactive group such as haloaceyl, maleiamide, etc., via a thioether to a second molecule which contains an amino group via an amide bond. These linkers are flexible with regard to the order of attachment, i.e., the thioether can be formed first or last.

In embodiments in which a non-immunostimulatory polynucleotide and antigen are coupled by adsorption onto a surface, the surface may be in the form of a carrier particle (for example, a nanoparticle or a microparticle) made with either an inorganic or organic core. Examples of such nanoparticles include, but are not limited to, nanocrystalline particles, nanoparticles made by the polymerization of alkylcyanoacrylates and nanoparticles made by the polymerization of methylidene malonate. Additional surfaces to which a non-immunostimulatory polynucleotide and antigen may be adsorbed include, but are not limited to, activated carbon particles and protein-ceramic nanoplates. Other examples of carrier particles are provided herein.

Adsorption of polynucleotides and polypeptides to a surface for the purpose of delivery of the adsorbed molecules to cells is well known in the art. See, for example, Douglas et al. (1987) Crit. Rev. Ther. Drug. Carrier Syst. 3:233-261; Hagiwara et al. (1987) In Vivo 1:241-252; Bousquet et al. (1999) Pharm. Res. 16:141-147; and Kossovsky et al., U.S. Pat. No. 5,460,831. Preferably, the material comprising the adsorbent surface is biodegradable. Adsorption of a non-immunostimulatory polynucleotide and/or antigen to a surface may occur through non-covalent interactions, including ionic and/or hydrophobic interactions.

In general, characteristics of carriers such as nanoparticles, such as surface charge, particle size and molecular weight, depend upon polymerization conditions, monomer concentration and the presence of stabilizers during the polymerization process (Douglas et al., 1987). The surface of carrier particles may be modified, for example, with a surface coating, to allow or enhance adsorption of the polynucleotide and/or antigen. Carrier particles with adsorbed polynucleotide and/or antigen may be further coated with other substances. The addition of such other substances may, for example, prolong the half-life of the particles once administered to the subject and/or may target the particles to a specific cell type or tissue, as described herein.

Nanocrystalline surfaces to which a non-immunostimulatory polynucleotide and antigen may be adsorbed have been described (see, for example, U.S. Pat. No. 5,460,831). Nanocrystalline core particles (with diameters of 1 μm or less) are coated with a surface energy modifying layer that promotes adsorption of polypeptides, polynucleotides and/or other pharmaceutical agents. As described in U.S. Pat. No. 5,460,831, for example, a core particle is coated with a surface that promotes adsorption of an oligonucleotide and is subsequently coated with an antigen preparation, for example, in the form of a lipid-antigen mixture.

Another adsorbent surface are nanoparticles made by the polymerization of alkylcyanoacrylates. Alkylcyanoacrylates can be polymerized in acidified aqueous media by a process of anionic polymerization. Depending on the polymerization conditions, the small particles tend to have sizes in the range of 20 to 3000 nm, and it is possible to make nanoparticles specific surface characteristics and with specific surface charges (Douglas et al., 1987). For example, oligonucleotides may be adsorbed to polyisobutyl- and polyisohexlcyanoacrylate nanoparticles in the presence of hydrophobic cations such as tetraphenylphosphonium chloride or quaternary ammonium salts, such as cetyltrimethyl ammonium bromide. Oligonucleotide adsorption on these nanoparticles appears to be mediated by the formation of ion pairs between negatively charged phosphate groups of the nucleic acid chain and the hydrophobic cations. See, for example, Lambert et al. (1998) Biochimie 80:969-976, Chavany et al. (1994) Pharm. Res. 11:1370-1378; Chavany et al. (1992) Pharm. Res. 9:441-449. Polypeptides may also be adsorbed to polyalkylcyanoacrylate nanoparticles. See, for example, Douglas et al., 1987; Schroeder et al. (1998) Peptides 19:777-780.

Another adsorbent surface are nanoparticles made by the polymerization of methylidene malonate. For example, as described in Bousquet et al., 1999, polypeptides adsorbed to poly(methylidene malonate 2.1.2) nanoparticles appear to do so initially through electrostatic forces followed by stabilization through hydrophobic forces.

Microcarriers useful in the invention are less than about 150, 120 or 100 μm in size, more commonly less than about 50-60 μmin size, preferably less than about 10 μm in size, and are insoluble in pure water. Microcarriers used in the invention are preferably biodegradable, although nonbiodegradable microcarriers are acceptable. Microcarriers are commonly solid phase, such as “beads” or other particles, although liquid phase microcarriers such as oil in water emulsions comprising a biodegradable polymers or oils are also contemplated. A wide variety of biodegradable and nonbiodegradable materials acceptable for use as microcarriers are known in the art.

Microcarriers for use in the NISC compositions or methods of the invention are generally less than about 10 μm in size (e.g., have an average diameter of less than about 10 μm, or at least about 97% of the particles pass through a 10 μm screen filter), and include nanocarriers (i.e., carriers of less than about 1 μm size). Preferably, microcarriers are selected having sizes within an upper limit of about 9, 7, 5, 2, or 1 μm or 900, 800, 700, 600, 500, 400, 300, 250, 200, or 100 nm and an independently selected lower limit of about 4, 2, or 1 μm or about 800, 600, 500, 400, 300, 250, 200, 150, 100, 50, 25, or 10 nm, where the lower limit is less than the upper limit. In some embodiments, the microcarriers have a size of about 1.0-1.5 μm, about 1.0-2.0 μm or about 0.9-1.6 μm. In certain preferred embodiments, the microcarriers have a size of about 10 nm to about 5 μm or about 25 nm to about 4.5 μm, about 1 μm, about 1.2 μm, about 1.4 μm, about 1.5 μm, about 1.6 μm, about 1.8 μm, about 2.0 μm, about 2.5 μm or about 4.5 μm. When the microcarriers are nanocarriers, preferred embodiments include nanocarriers of about 25 to about 300 nm, 50 to about 200 nm, about 50 nm or about 200 nm.

Solid phase biodegradable microcarriers may be manufactured from biodegradable polymers including, but not limited to: biodegradable polyesters, such as poly(lactic acid), poly(glycolic acid), and copolymers (including block copolymers) thereof, as well as block copolymers of poly(lactic acid) and poly(ethylene glycol); polyorthoesters such as polymers based on 3,9-diethylidene-2,4,8,10-tetraoxaspiro[5.5]undecane (DETOSU); polyanhydrides such as poly(anhydride) polymers based on relatively hydrophilic monomers such as sebacic acid; polyanhydride imides, such as polyanhydride polymers based on sebacic acid-derived monomers incorporating amino acids (i.e., linked to sebacic acid by imide bonds through the amino-terminal nitrogen) such as glycine or alanine; polyanhydride esters; polyphosphazenes, especially poly(phosphazenes) which contain hydrolysis-sensitive ester groups which can catalyze degradation of the polymer backbone through generation of carboxylic acid groups (Schacht et al., (1996) Biotechnol. Bioeng. 1996:102); and polyamides such as poly(lactic acid-co-lysine).

A wide variety of nonbiodegradable materials suitable for manufacturing microcarriers are also known, including, but not limited to polystyrene, polypropylene, polyethylene, silica, ceramic, polyacrylamide, dextran, hydroxyapatite, latex, gold, and ferromagnetic or paramagnetic materials. Certain embodiments exclude gold, latex, and/or magnetic beads. In certain embodiments, the microcarriers may be made of a first material (e.g., a magnetic material) encapsulated with a second material (e.g., polystyrene).

Solid phase microspheres are prepared using techniques known in the art. For example, they can be prepared by emulsion-solvent extraction/evaporation technique. Generally, in this technique, biodegradable polymers such as polyanhydrates, poly(alkyl-β-cyanoacrylates) and poly(β-hydroxy esters), for example, poly(lactic acid), poly(glycolic acid), poly(D,L-lactic-co-glycolic acid) and poly(caprolactone), are dissolved in a suitable organic solvent, such as methylene chloride, to constitute the dispersed phase (DP) of emulsion. DP is emulsified by high-speed homogenization into excess volume of aqueous continuous phase (CP) that contains a dissolved surfactant, for example, polyvinylalcohol (PVA) or polyvinylpirrolidone (PVP). Surfactant in CP is to ensure the formation of discrete and suitably-sized emulsion droplet. The organic solvent is then extracted into the CP and subsequently evaporated by raising the system temperature. The solid microparticles are then separated by centrifugation or filtration, and dried, for example, by lyophilization or application of vacuum, before storing at 4° C.

Physico-chemical characteristics such as mean size, size distribution and surface charge of dried microspheres may be determined. Size characteristics are determined, for example, by dynamic light scattering technique and the surface charge was determined by measuring the zeta potential.

Covalently bound non-immunostimulatory polynucleotide and antigen to a microcarrier may be linked using any covalent crosslinking technology described herein or known in the art. A wide variety of crosslinking technologies are known in the art, and include crosslinkers reactive with amino, carboxyl and sulfhydryl groups. As will be apparent to one of skill in the art, the selection of a crosslinking agent and crosslinking protocol will depend on the configuration of the non-immunostimulatory polynucleotide, antigen, and microcarrier as well as the desired final configuration of the NISC. The crosslinker may be either homobifunctional or heterobifunctional. When a homobifunctional crosslinker is used, the crosslinker exploits the same moiety on the non-immunostimulatory polynucleotide (or antigen) and microcarrier (e.g., an aldehyde crosslinker may be used to covalently link a polynucleotide (or antigen) and microcarrier where both the polynucleotide (or antigen) and microcarrier comprise one or more free amines). Heterobifunctional crosslinkers utilize different moieties on the non-immunostimulatory polynucleotide (or antigen) and microcarrier, (e.g., a maleimido-N-hydroxysuccinimide ester may be used to covalently link a free sulfhydryl on the polynucleotide and a free amine on the microcarrier), and are preferred to minimize formation of inter-microcarrier bonds. The crosslinker may incorporate a “spacer” arm between the reactive moieties, or the two reactive moieties in the crosslinker may be directly linked.

In one embodiment, the polynucleotide portion comprises at least one free sulfhydryl (e.g., provided by a 5′-thiol modified base or linker) for crosslinking to the microcarrier, while the microcarrier comprises free amine groups. A heterobifunctional crosslinker reactive with these two groups (e.g., a crosslinker comprising a maleimide group and a NHS-ester), such as succinimidyl 4-(N-maleimidomethyl)cyclohexane-1-carboxylate is used to activate the microcarrier, then covalently crosslink the polynucleotide to form the polynucleotide/microcarrier complex.

NISCs comprising microcarriers may involve linkages by any non-covalent binding or interaction, including ionic (electrostatic) bonds, hydrophobic interactions, hydrogen bonds, van der Waals attractions, or a combination of two or more different interactions.

Preferred non-covalent NISC microcarrier complexes are typically complexed by hydrophobic or electrostatic (ionic) interactions, or a combination thereof, (e.g., through base pairing between a non-immunostimulatory polynucleotide and a polynucleotide bound to a microcarrier as a binding pair). Due to the hydrophilic nature of the backbone of polynucleotides, NISC complexes which rely on hydrophobic interactions to form the complex generally require modification of the polynucleotide portion of the complex to incorporate a highly hydrophobic moiety. Preferably, the hydrophobic moiety is biocompatible, nonimmunogenic, and is naturally occurring in the individual for whom the composition is intended (e.g., is found in mammals, particularly humans). Examples of preferred hydrophobic moieties include lipids, steroids, sterols such as cholesterol, and terpenes. The method of linking the hydrophobic moiety to the polynucleotide or antigen will, of course, depend on the configuration of the polynucleotide or antigen and the identity of the hydrophobic moiety. The hydrophobic moiety may be added at any convenient site in the polynucleotide, preferably at either the 5′ or 3′ end; in the case of addition of a cholesterol moiety to a polynucleotide, the cholesterol moiety is preferably added to the 5′ end of the polynucleotide, using conventional chemical reactions (see, for example, Godard et al. (1995) Eur. J. Biochem. 232:404-410). Preferably, microcarriers for use in NISC complexes linked by hydrophobic bonding are made from hydrophobic materials, such as oil droplets or hydrophobic polymers, although hydrophilic materials modified to incorporate hydrophobic moieties may be utilized as well.

Non-covalent NISC complexes bound by electrostatic binding typically exploit the highly negative charge of the polynucleotide backbone. Accordingly, microcarriers for use in non-covalently bound NISC complexes are generally positively charged (cationic) at physiological pH (e.g., about pH 6.8-7.4). The microcarrier may intrinsically possess a positive charge, but microcarriers made from compounds not normally possessing a positive charge may be derivatized or otherwise modified to become positively charged (cationic). For example, the polymer used to make the microcarrier may be derivatized to add positively charged groups, such as primary amines. Alternately, positively charged compounds may be incorporated in the formulation of the microcarrier during manufacture (e.g., positively charged surfactants may be used during the manufacture of poly(lactic acid)/poly(glycolic acid) copolymers to confer a positive charge on the resulting microcarrier particles).

For example, to prepare cationic microspheres, cationic lipids or polymers, for example, 1,2-dioleoyl-1,2,3-trimethylammoniopropane (DOTAP), cetyltrimethylammonium bromide (CTAB) or polylysine, are added either to DP or CP, as per their solubility in these phases.

NISCs can be preformed by adsorption onto cationic microspheres by incubation of polynucleotide, antigen, and the particles, preferably in an aqueous admixture. Such incubation may be carried out under any desired conditions, including ambient (room) temperature (e.g., approximately 20° C.) or under refrigeration (e.g., 4° C.). Because cationic microspheres and polynucleotides associate relatively quickly, the incubation may be for any convenient time period, such as 5, 10, 15 minutes or more, including overnight and longer incubations. For example, polynucleotides can be adsorbed onto the cationic microspheres by overnight aqueous incubation of polynucleotide and the particles at 4° C. However, because cationic microspheres and polynucleotides spontaneously associate, the NISC can be formed by simple co-administration of the polynucleotide, antigen, and the microcarrier. Microspheres may be characterized for size and surface charge before and after polynucleotide association. Selected batches may then evaluated for activity against suitable controls in, for example, APCs. The formulations may also evaluated in suitable animal models.

Non-covalent NISCs linked by nucleotide base pairing may be produced using conventional methodologies. Generally, base-paired NISC complexes are produced using a microcarrier comprising a bound, preferably a covalently bound, polynucleotide (the “capture polynucleotide”) that is at least partially complementary to the non-immunostimulatory polynucleotide. The segment of complementarity between the non-immunostimulatory polynucleotide and the capture nucleotide is preferably at least 6, 8, 10 or 15 contiguous base pairs, more preferably at least 20 contiguous base pairs. The capture nucleotide may be bound to the microcarrier by any method known in the art, and may be covalently bound to the non-immunostimulatory polynucleotide at the 5′ or 3′ end. In some embodiments, a non-immunostimulatory polynucleotide comprising a 5′-GGGG-3′ sequence will retain this portion of the sequence as single-stranded.

In other embodiments, a binding pair may be used to link the non-immunostimulatory polynucleotide and/or antigen and microcarrier in a NISC. The binding pair may be a receptor and ligand, an antibody and antigen (or epitope), or any other binding pair which binds at high affinity (e.g., Kd less than about 10⁻⁸). One type of preferred binding pair is biotin and streptavidin or biotin and avidin, which form very tight complexes. When using a binding pair to mediate NISC binding, the non-immunostimulatory polynucleotide and/or antigen is derivatized, typically by a covalent linkage, with one member of the binding pair, and the microcarrier is derivatized with the other member of the binding pair. Mixture of the derivatized compounds results in NISC formation.

Methods of the Invention

The invention provides methods of regulating an immune response in an individual, prefereably a mammal, more preferably a human, comprising administering to the individual a non-immunostimulatory conjugate (NISC) as described herein. Methods of immunoregulation provided by the invention include those that suppress and/or inhibit an unwanted immune response to an antigen, including, but not limited to, an autoimmune response, an allergic response, and similarly aberrant immune responses, for example, those found in celiac disease. The invention also provides methods for generation of antigen-specific T regulatory cells and methods for inhibiting Th1 and/or Th2 cell differentiation.

The invention also provides methods for ameliorating symptoms associated with unwanted immune activation, including, but not limited to, symptoms associated with autoimmunity, symptoms associated with allergy, symptoms associated with similarly aberrant immune responses, such as in celiac disease, and symptoms associated with alloimmunity. Accordingly, the invention also provides methods for aiding in transplantation, such as reducing graft rejection and/or graft-versus-host (GVH) disease.

As demonstrated herein, linkage of an non-immunostimulatory oligonucleotide to an antigen (NISC) leads to an increased uptake of the antigen as compared to administration of a mixture of the antigen and oligonucleotide. Despite the antigen uptake, little or no DC maturation was stimulated by the NISC composition. By contrast, incubation with the immunostimulatory oligonucleotide—antigen conjugate resulted in both antigen uptake by the DCs and stimulation of DC maturation.

The NISC is administered in an amount sufficient to regulate an immune response to an antigen. As described herein, regulation of an immune response may be humoral and/or cellular, and is measured using standard techniques in the art and as described herein.

In certain embodiments, the individual suffers from a disorder associated with unwanted immune activation, such as allergic disease or condition, allergy and asthma. An individual having an allergic disease or asthma is an individual with a recognizable symptom of an existing allergic disease or asthma.

In certain embodiments, the individual suffers from a disorder associated with unwanted immune activation, such as autoimmune disease. An individual having an autoimmune disease is an individual with a recognizable symptom of an existing autoimmune disease.

Autoimmune diseases can be divided in two broad categories: organ-specific and systemic. Autoimmune diseases include, without limitation, rheumatoid arthritis (RA), systemic lupus erythematosus (SLE), type I diabetes mellitus, type II diabetes mellitus, multiple sclerosis (MS), immune-mediated infertility such as premature ovarian failure, scleroderma, Sjogren's disease, vitiligo, alopecia (baldness), polyglandular failure, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (HCV), hypopituitarism, graft-versus-host disease (GvHD), myocarditis, Addison's disease, autoimmune skin diseases, uveitis, pernicious anemia, and hypoparathyroidism.

Autoimmune diseases may also include, without limitation, Hashimoto's thyroiditis, Type I and Type II autoimmune polyglandular syndromes, paraneoplastic pemphigus, bullus pemphigoid, dermatitis herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, glomerulonephritis, and cryopathies. These conditions are well known in the medical arts and are described, for example, in Harrison's Principles of Internal Medicine, 14th ed., Fauci A S et al., eds., New York: McGraw-Hill, 1998.

The systemic disease SLE is characterized by the presence of antibodies to antigens that are abundant in nearly every cell, such as anti-chromatin antibodies, anti-spliceosome antibodies, anti-ribosome antibodies and anti-DNA antibodies. Consequently, the effects of SLE are seen in a variety of tissues, such as the skin and kidneys. Autoreactive T cells also play a role in SLE. For example, studies in a murine lupus model have shown that non-DNA nucleosomal antigens, e.g. histones, stimulate autoreactive T cells that can drive anti-DNA producing B cells. Increased serum levels of IFN-α has been observed in SLE patients and shown to correlate with both disease activity and severity, including fever and skin rashes, as well as essential markers associated with the disease process (e.g., anti-dsDNA antibody titers).

In certain embodiments, an individual is at risk of developing an autoimmune disease and an NISC is administered in an ammount effective to delay or prevent the autoimmune disease. Individuals at risk of developing an autoimmune disease includes, for example, those with a genetic or other predisposition toward developing an autoimmune disease. In humans, susceptibility to particular autoimmune diseases is associated with HLA type with some being linked most strongly with particular MHC class II alleles and others with particular MHC class I alleles. For example, ankylosing spondylitis, acute anterior uveitis, and juvenile rheumatoid arthritis are associated with HLA-B27, Goodpasture's syndrome and MS are associated with HLA-DR2, Grave's disease, myasthenia gravis and SLE are associated with HLA-DR3, rheumatoid arthritis and pemphigus vulgaris are associated with HLA-DR4 and Hashimoto's thyroiditis is associated with HLA-DRS. Other genetic predispositions to autoimmune diseases are known in the art and an individual can be examined for existence os such predispositions by assays and methods well known in the art. Accordingly, in some instances, an individual at risk of developing an autoimmune can be identified.

As described herein, NISCs of the invention are taken up by plasmacytoid dendritic cells (DCs) and/or antigen presenting cells (APCs) without the oligonucleotide portion of the NISC inducing or promoting activation or maturation of the DC or the APC. By preventing such activation or maturation, the NISC may also prevent production of a cytokine, including, but not limited to, IL-6, IL-12, TNF-α, and/or IFN-α, and may prevent B cell proliferation.

Animal models for the study of autoimmune disease are known in the art. For example, animal models which appear most similar to human autoimmune disease include animal strains which spontaneously develop a high incidence of the particular disease. Examples of such models include, but are not limited to, the nonobeses diabetic (NOD) mouse, which develops a disease similar to type 1 diabetes, and lupus-like disease prone animals, such as New Zealand hybrid, MRL-Fas^{1pr} and BXSB mice. Animal models in which an autoimmune disease has been induced include, but are not limited to, experimental autoimmune encephalomyelitis (EAE), which is a model for multiple sclerosis, collagen-induced arthritis (CIA), which is a model for rheumatoid arthritis, and experimental autoimmune uveitis (EAU), which is a model for uveitis. Animal models for autoimmune disease have also been created by genetic manipulation and include, for example, IL-2/IL-10 knockout mice for inflammatory bowel disease, Fas or Fas ligand knockout for SLE, and IL-1 receptor antagonist knockout for rheumatoid arthritis.

Accordingly, animal models standard in the art are available for the screening and/or assessment for activity and/or effectiveness of the methods and compositions of the invention for the treatment of autoimmune disorders.

The methods of the invention may be practiced in combination with other therapies which make up the standard of care for the disorder or condition, such as administration of anti-rejection agents and immune suppression agents. For example, anti-inflammatory drugs, anti-malarials, steroids (such as cortisone), and cytotoxic chemotherapies are used in the treatment of SLE.

Tolerance to autoantigens and autoimmune disease is achieved by a variety of mechanisms including negative selection of self-reactive T cells in the thymus and mechanisms of peripheral tolerance for those autoreactive T cells that escape thymic deletion and are found in the periphery. Examples of mechanisms that provide peripheral T cell tolerance include “ignorance” of self antigens, anergy or unresponsiveness to autoantigen, cytokine immune deviation, and activation-induced cell death of self-reactive T cells. In addition, regulatory T cells have been shown to be involved in mediating peripheral tolerance. See, for example, Walker et al. (2002) Nat. Rev. Immunol. 2:11-19; Shevach et al. (2001) Immunol. Rev. 182:58-67. In some situations, peripheral tolerance to an autoantigen is lost (or broken) and an autoimmune response ensues. For example, in an animal model for EAE, activation of antigen presenting cells (APCs) through TLR innate immune receptors was shown to break self-tolerance and result in the induction of EAE (Waldner et al. (2004) J. Clin. Invest. 113:990-997).

Accordingly, in some embodiments, the invention provides methods for increasing antigen presentation while suppressing or reducing TLR7/8, TLR9, and/or TLR 7/8/9 dependent cell stimulation. As described herein, administration of particular NISCs results in antigen presentation by DCs or APCs while suppressing the TLR 7/8, TLR9, and/or TLR7/8/9 dependent cell responses associated with immunostimulatory polynucleotides. Such suppression may include decreased levels of one or more TLR-associated cytokines. IRPs appropriate for use in suppressing TLR9 dependent cell stimulation are those IRP that inhibit or suppress cell responses associated with TLR9.

Administration and Assessment

The NISC can be administered in combination with other pharmaceutical agents, as described herein, and can be combined with a physiologically acceptable carrier thereof (and as such the invention includes these compositions). The NISC may be any of those described herein.

As with all compositions for modulation of an immune response, the effective amounts and method of administration of the particular NISC formulation can vary based on the individual, what condition is to be treated and other factors evident to one skilled in the art. Factors to be considered include whether or not the NISC will be administered with or covalently attached to a delivery molecule, route of administration and the number of doses to be administered. Such factors are known in the art and it is well within the skill of those in the art to make such determinations without undue experimentation. A suitable dosage range is one that provides the desired regulation of immune response (e.g., an increase in antigen-specific regulatory T cells). When induction or promotion of peripheral self-tolerance is desired, a suitable dosage range is one that provides the desired induction or promotion of peripheral self-tolerance. Generally, dosage is determined by the amount of oligonucleotide in the NISC administered to the patient, rather than the overall quantity of NISC-containing composition administered. Useful dosage ranges of the oligonucleotide of the NISC, given in amounts of oligonucleotide delivered, may be, for example, from about any of the following: 0.5 to 10 mg/kg, 1 to 9 mg/kg, 2 to 8 mg/kg, 3 to 7 mg/kg, 4 to 6 mg/kg, 5 mg/kg, 1 to 10 mg/kg, 5 to 10 mg/kg. The absolute amount given to each patient depends on pharmacological properties such as bioavailability, clearance rate and route of administration.

The effective amount and method of administration of the particular NISC formulation can vary based on the individual patient, desired result and/or type of disorder, the stage of the disease and other factors evident to one skilled in the art. The route(s) of administration useful in a particular application are apparent to one of skill in the art. Routes of administration include but are not limited to topical, dermal, transdermal, transmucosal, epidermal, parenteral, gastrointestinal, and naso-pharyngeal and pulmonary, including transbronchial and transalveolar. A suitable dosage range is one that provides sufficient NISC-containing composition to attain a tissue concentration of about 1-50 μM as measured by blood levels. The absolute amount given to each patient depends on pharmacological properties such as bioavailability, clearance rate and route of administration.

As described herein, tissues in which unwanted immune activation is occurring or is likely to occur are preferred targets for the NISC. Thus, skin, lymph nodes, spleen, bone marrow, and blood are preferred sites of NISC administration.

The present invention provides NISC formulations suitable for topical application including, but not limited to, physiologically acceptable implants, ointments, creams, rinses and gels. Exemplary routes of dermal administration are those which are least invasive such as transdermal transmission, epidermal administration and subcutaneous injection.

Transdermal administration is accomplished by application of a cream, rinse, gel, etc. capable of allowing the NISC to penetrate the skin and enter the blood stream. Compositions suitable for transdermal administration include, but are not limited to, pharmaceutically acceptable suspensions, oils, creams and ointments applied directly to the skin or incorporated into a protective carrier such as a transdermal device (so-called “patch”). Examples of suitable creams, ointments etc. can be found, for instance, in the Physician's Desk Reference. Transdermal transmission may also be accomplished by iontophoresis, for example using commercially available patches which deliver their product continuously through unbroken skin for periods of several days or more. Use of this method allows for controlled transmission of pharmaceutical compositions in relatively great concentrations, permits infusion of combination drugs and allows for contemporaneous use of an absorption promoter.

Parenteral routes of administration include but are not limited to electrical (iontophoresis) or direct injection such as direct injection into a central venous line, intravenous, intramuscular, intraperitoneal, intradermal, or subcutaneous injection. Formulations of NISC suitable for parenteral administration are generally formulated in USP water or water for injection and may further comprise pH buffers, salts bulking agents, preservatives, and other pharmaceutically acceptable excipients. NISC for parenteral injection may be formulated in pharmaceutically acceptable sterile isotonic solutions such as saline and phosphate buffered saline for injection.

Gastrointestinal routes of administration include, but are not limited to, ingestion and rectal routes and can include the use of, for example, pharmaceutically acceptable powders, pills or liquids for ingestion and suppositories for rectal administration.

Naso-pharyngeal and pulmonary administration include are accomplished by inhalation, and include delivery routes such as intranasal, transbronchial and transalveolar routes. The invention includes formulations of NISC suitable for administration by inhalation including, but not limited to, liquid suspensions for forming aerosols as well as powder forms for dry powder inhalation delivery systems. Devices suitable for administration by inhalation of NISC formulations include, but are not limited to, atomizers, vaporizers, nebulizers, and dry powder inhalation delivery devices.

As is well known in the art, solutions or suspensions used for the routes of administration described herein can include any one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As is well known in the art, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

As is well known in the art, sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The above-mentioned compositions and methods of administration are meant to describe but not limit the methods of administering the formulations of NISCs of the invention. The methods of producing the various compositions and devices are within the ability of one skilled in the art and are not described in detail here.

Analysis (both qualitative and quantitative) of the activity of an NISC in suppression of an unwanted immune response can be by any method described herein or known in the art, including, but not limited to, measuring generation of antigen-specific regulatory T cells, measuring an induction or promotion in antigen tolerance, measuring suppression or a decrease in proliferation of specific cell populations such as B cells, measuring suppression or a decrease in Th1 cell and/or Th2 cell differentiation, measuring supression of maturation of specific cell populations such as dendritic cells (including plasmacytoid dendritic cells) and T cells (including decrease in Th1 and/or Th2 cell differentiation), and measuring suppression in production of cytokines such as, but not limited to, IFN-α, TNF-α, IL-6, and/or IL-12. Measurement of numbers of specific types of cells can be achieved, for example, with fluorescence-activated cell sorting (FACS). Measurement of maturation of particular populations of cells can be achieved by determining expression of markers, for example, cell surface markers, specific for particular stage of cell maturation. Cell marker expression can be measured, for example, by measuring RNA expression or measuring cell surface expression of the particular marker by, for example, FACS analysis. Measuring maturation of dendritic cells can be performed for instance as described in Hartmann et al. (1999) Proc. Natl. Acad. Sci. USA 96:9305-9310. Cytokine concentrations can be measured, for example, by ELISA. These and other assays to evaluate suppression of an immune response, including an innate immune response, are well known in the art.

Kits of the Invention

The invention provides kits. In certain embodiments, the kits of the invention generally comprise one or more containers comprising any NISC as described herein. The kits may further comprise a suitable set of instructions, generally written instructions, relating to the use of the NISC for any of the methods described herein (e.g., suppression of a response to an unwanted immune response, suppression of an autoimmune response, induction or promotion of peripheral tolerance, ameliorating one or more symptoms of an autoimmune disease, ameliorating a symptom of allergy, stimulating generation of antigen-specific regulatory T cells).

The kits may comprise NISC packaged in any convenient, appropriate packaging. For example, if the NISC is a dry formulation (e.g., freeze dried or a dry powder), a vial with a resilient stopper is normally used, so that the NISC may be easily resuspended by injecting fluid through the resilient stopper. Ampoules with non-resilient, removable closures (e.g., sealed glass) or resilient stoppers are most conveniently used for liquid formulations of NISC. Also contemplated are packages for use in combination with a specific device, such as an inhaler, nasal administration device (e.g., an atomizer), a syringe or an infusion device such as a minipump.

The instructions relating to the use of NISC generally include information as to dosage, dosing schedule, and route of administration for the intended method of use. The containers of NISC may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, kits of the invention comprise materials for production of NISC as complexes for administration, for example, encapsulation material, microcarrier complex material and so on. Generally, the kit inlcudes separate containers of NISC and the complex material(s). The NISC and complexes are preferably supplied in a form which allows formation of NISC-complex upon mixing of the supplied NISC and complex material. This configuration is preferred when the NISC-complex is linked by non-covalent bonding. This configuration is also preferred when the NISC-complex are to be crosslinked via a heterobifunctional crosslinker; either NISC or the complex is supplied in an “activated” form (e.g., linked to the heterobifunctional crosslinker such that a moiety reactive with the NISC is available).

The following Examples are provided to illustrate, but not limit, the invention.

### EXAMPLES

### Example 1

### Antigen Uptake and Dendritic Cell Maturation

The effect of conjugation of an oligonucleotide to an antigen on antigen uptake and presentation by dendritic cells was examined. Fluorescently-labeled ovalbumin (OVA linked to Alexa 647) was added to a culture of murine dendritic cells: a) in a mixture with an immunostimulatory oligonucleotide (5′-TGACTGTGAACGTTCGAGATGA-3′ (1018) SEQ ID NO:72), b) conjugated to an immunostimulatory oligonucleotide (1018), or c) conjugated to non-immunostimulatory oligonucleotide ((5′-TGACTGTGAACCTTAGAGATGA-3′ (1040) SEQ ID NO: 73). Alexa 647 fluorescence incorporated into the dendritic cells (DC) was then evaluated by flow cytometry using standard methods.

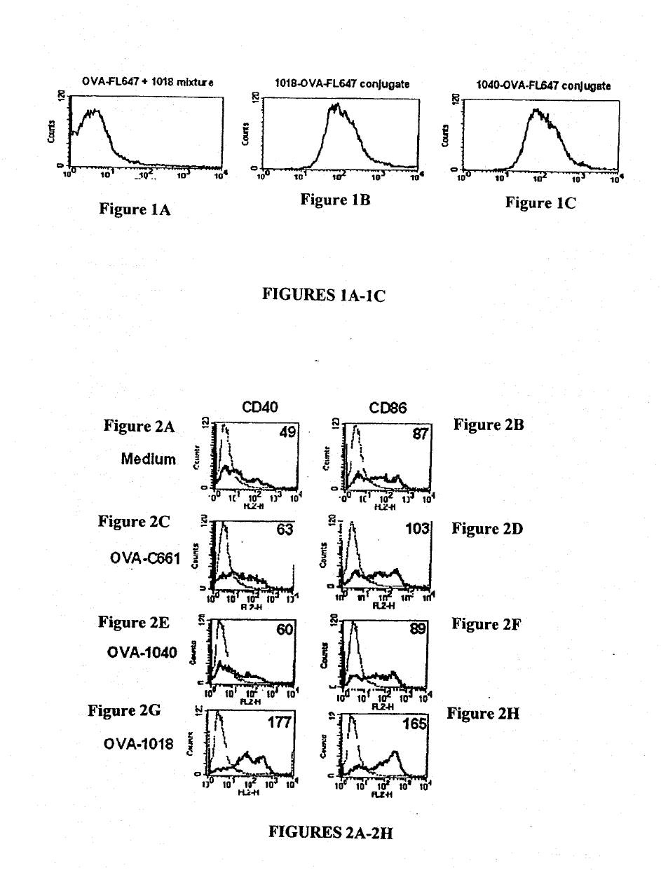

As shown in FIGS. 1A-1C, both the ISS-conjugate (1018-OVA, center graph) and the NISC (1040-OVA, right graph) treated DC had increased fluorescence as compared with OVA mixed, but not conjugated to, 1018 (mixture, left graph). Thus, conjugation to an immunostimulatory oligonucleotide or to a non-immunostimulatory oligonucleotide promotes uptake of the antigen by dendritic cells.

Maturation of the murine dendritic cells after incubation with the oligonucleotide/antigen compositions was evaluated by the up-regulation of co-stimulatory molecules such as CD40, CD80 and CD86. As shown in FIGS. 2-2H, cell maturation markers after incubation with NISCs (OVA-C661 (5′-TGCTTGCAAGCTTGCAAGCA-3′) SEQ ID NO: 27 and OVA-1040 (5′-TGACTGTGAACCTTAGAGATGA-3′) SEQ ID NO: 73) were similar to those found on the cells incubated in cell medium alone. Thus, the NISCs induced little to no dendritic cell maturation. In contrast, the immunostimulatory oligonucleotide conjugate, OVA-1018 (5′-TGACTGTGAACGTTCGAGATGA-3′ SEQ ID NO: 72) greatly induced the level of maturation markers on the cells.

Human plasmacytoid dendritic cells (PDCs) were isolated using procedures descibed in Marshall et al. (2003) J. Leukoc Biol. 73:781-92. PDCs were dispensed and incubated with conjugates or oligonucleotides for 24 hours. Maturation markers were then measured on the cells and the results are shown in Table 2.

TABLE 2
Maturation marker expression (mean fluorescence instensity)

| Cells/culture conditions | CD80 | CD86 | CD40 |
|---|---|---|---|
| Freshly isolated | 4 | 10 | 4 |
| Medium | 5 | 5 | 9 |
| SEQ ID NO: 1018 | 51 | 25 | 50 |
| SEQ ID NO: 1040 | 26 | 14 | 37 |
| OVA-SEQ ID NO: 1018 conjugate | 53 | 31 | 53 |
| OVA-SEQ ID NO: 1040 conjugate | 21 | 14 | 32 |

As shown in Table 2 and FIGS. 2A-2H, similar results were obtained with PDC isolated from human blood and with DC from mice. Thus, despite causing very high levels of uptake, the NISC induces only a low level of dendritic cell maturation.

### Example 2

### In Vivo Responses to NISC and NISC-Activated DC

In vivo the tolerogenic properties of NISC-activated DC are evaluated in a model in which the lung is used an the target compartment as described by Lambrecht et al. (2000, J. Immunol. 164:2937-2946). This robust system allows characterization of the response to NISC-activated DC. Ovalbumin (OVA) is used as an antigen is these assays.

10⁶ NISC-activated DC are injected in the trachea of anesthetized BALB/c mice using a 25 gauge metal catheter. Other groups of mice receive DC pulsed with an immunostimulatory oligonucleotide-OVA conjugate or OVA alone. Forty-eight hours before the DC injection, 25×10⁶ purified carboxyfluorescein diacetate succinimidyl ester (CFSE)-labeled naïve OVA-specific CD4+ T cells are adoptively transferred in the mice. Following encounter of the T cells with the DC, the T cells are harvested from the lungs of the animals and analyzed for proliferation or restimulated in vitro with splenocytes and OVA to analyze their cytokine response by costaining for KJ1-26 and intracellular staining for cytokine content.

In vivo the tolerogenic properties of NISC-activated DC are also evaluated in a model in which the activated DC are injected into the footpad of the animal and T cell responses in the draining lymph nodes are assessed.

Both the direct response to NISC injection and its effect in regulating subsequent inoculation with OVA or with an unrelated antigen (e.g., hen egg white lysozyme, HEL) are analyzed in mice. To evaluate the direct response to NISC(OVA), mice are injected once a week for two weeks (D0 and D7) intraperitoneally (i.p.) with NISC (OVA), immunostimulatory oligonucleotide-OVA conjugate, OVA-Alum or PBS. At day 14, mice are bled to measure antibody response (OVA specific IgG1, IgE and IgG2a) and challenged in the footpad with OVA.

To evaluate the effect of NISC on subsequent Th1 or Th2 responses, mice are injected i.p. once a week for two weeks (D0 and D7) with NISC(OVA), OVA alone or PBS. At day 14, mice are bled to measure antibody response and receive either OVA-Alum i.p. or OVA-CFA (complete Fruend's adjuvant) subcutaneously (s.c.). A week later, at day 21, mice are bled to measure antibody response and challenged with OVA. At day 25, antibody and recall responses are measured. Alternatively, subsequent immunization is conducted with an unrelated antigen, such as HEL, in order to address the issue of antigen specificity. Interference of the subsequent immunization and dampening of the Th1/Th2 response in response to the pretreatment of with NISC are indications of induction of peripheral tolerance.

To determine whether antigen-specific tolerance induced by NISC activation of DC in vivo would be effective in a therapeutic setting, the effect of NISC treatment on an already established Th1 or Th2 response is evaluated.

Mice are first injected with OVA-Alum i.p (Th2 polarizing conditions) or OVA-CFA s.c. (Th1 driving situation). Starting 2 weeks later, mice receive two immunizations with NISC(OVA), OVA alone or PBS i.p (D14 and D21). At day 35, two weeks after the last immunization, mice are challenged with OVA injection in the footpad of the animal. Four days later (D39), mice are sacrificed and the antibody response as well as cytokine response to a recall OVA stimulation of cells isolated from the draining lymph nodes is measured. A similar experiment using HEL as antigen is performed to determine whether NISC(OVA) induced peripheral tolerance is specific for the OVA antigen.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention.

1. A non-immunostimulatory conjugate (NISC) comprising a non-immunostimulatory polynucleotide linked to an antigen.

2. The conjugate according to claim 1, wherein the antigen is an autoantigen, an alloantigen or an allergen.

3. The conjugate according to claim 1, wherein the non-immunostimulatory polynucleotide comprises an immunoregulatory sequence (IRS).

4. The conjugate according to claim 3, wherein the IRS is a TLR9 class IRS, a TLR7/8 class IRS or a TLR7/8/9 IRS.

5. The conjugate according to claim 1, wherein the non-immunostimulatory polynucleotide comprises an antisense molecule or an aptamer.

6. A pharmaceutical composition comprising the conjugate according to claim 1 and pharmaceutically accepted excipient.

7. A method of inducing peripheral tolerance to an antigen in a subject comprising administering to a subject a composition comprising a non-immunostimulatory conjugate (NISC), wherein said NISC comprises a non-immunostimulatory polynucleotide linked to an antigen and wherein said composition is administered in an amount effective to induce peripheral tolerance to said antigen.

8. A method for ameliorating a symptom of an unwanted immune activation in a subject comprising administering to a subject in need thereof an effective amount of a non-immunostimulatory conjugate (NISC), wherein said NISC comprises a non-immunostimulatory polynucleotide linked to an antigen and wherein said unwanted immune activation is directed to said antigen.

9. The method according to claim 8, wherein said unwanted immune activation is an autoimmune response, an allergy, asthma, a graft-versus-host reaction or a graft rejection reaction.

10. A method for suppressing an autoimmune response in a subject comprising administering to a subject in need thereof a non-immunostimulatory conjugate (NISC) in an amount effective to suppress an autoimmune response, wherein said NISC comprises a non-immunostimulatory polynucleotide linked to an antigen, wherein said autoimmune response is directed to said antigen.

11. A method for suppressing a symptom of an autoimmune disease in a subject comprising administering to a subject in need thereof an effective amount of a non-immunostimulatory conjugate (NISC) wherein said NISC comprises a non-immunostimulatory polynucleotide linked to an antigen and wherein said autoimmune disease involves an immune response to said antigen.

12-15. (canceled)


SEQUENCE LISTING (90 sequences)

SEQ ID NO: 1 - DNA, 7 bp, Artificial Sequence
  Synthetic construct
nggggnn

SEQ ID NO: 2 - DNA, 7 bp, Artificial Sequence
  Synthetic construct
nggggnn

SEQ ID NO: 3 - DNA, 109 bp, Artificial Sequence
  Synthetic construct
ggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggnn

SEQ ID NO: 4 - DNA, 132 bp, Artificial Sequence
  Synthetic construct
nnnnnnnnnnnnnnnnnnnnnnnnnnnnnnnnnnnnnnnnnnnnnnnnnntccnnnnnnnnnnnnnnnnnnnnnnnnnnnnnnnnnnnnnnnnnnnnnnnnnnnnnnnnnnnnnnnnnnnnnnnnnggggnn

SEQ ID NO: 5 - DNA, 9 bp, Artificial Sequence
  Synthetic construct
nnnggggaa

SEQ ID NO: 6 - DNA, 118 bp, Artificial Sequence
  Synthetic construct
tgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcctggaggggttgt

SEQ ID NO: 7 - DNA, 17 bp, Artificial Sequence
  Synthetic construct
ttgacagcttgacagca

SEQ ID NO: 8 - DNA, 9 bp, Artificial Sequence
  Synthetic construct
tgcnnnnnn

SEQ ID NO: 9 - DNA, 107 bp, Artificial Sequence
  Synthetic construct
tgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 10 - DNA, 15 bp, Artificial Sequence
  Synthetic construct
tcctaacggggaagt

SEQ ID NO: 11 - DNA, 15 bp, Artificial Sequence
  Synthetic construct
tcctaagggggaagt

SEQ ID NO: 12 - DNA, 15 bp, Artificial Sequence
  Synthetic construct
tcctaacggggttgt

SEQ ID NO: 13 - DNA, 15 bp, Artificial Sequence
  Synthetic construct
tcctaacggggctgt

SEQ ID NO: 14 - DNA, 15 bp, Artificial Sequence
  Synthetic construct
tcctcaaggggctgt

SEQ ID NO: 15 - DNA, 15 bp, Artificial Sequence
  Synthetic construct
tcctcaaggggttgt

SEQ ID NO: 16 - DNA, 15 bp, Artificial Sequence
  Synthetic construct
tcctcatggggttgt

SEQ ID NO: 17 - DNA, 15 bp, Artificial Sequence
  Synthetic construct
tcctggaggggttgt

SEQ ID NO: 18 - DNA, 15 bp, Artificial Sequence
  Synthetic construct
tcctggaggggctgt

SEQ ID NO: 19 - DNA, 15 bp, Artificial Sequence
  Synthetic construct
tcctggaggggccat

SEQ ID NO: 20 - DNA, 15 bp, Artificial Sequence
  Synthetic construct
tcctggaggggtcat

SEQ ID NO: 21 - DNA, 15 bp, Artificial Sequence
  Synthetic construct
tccggaaggggaagt

SEQ ID NO: 22 - DNA, 15 bp, Artificial Sequence
  Synthetic construct
tccggaaggggttgt

SEQ ID NO: 23 - DNA, 15 bp, Artificial Sequence
  Synthetic construct
tcctggagnggttgt

SEQ ID NO: 24 - DNA, 22 bp, Artificial Sequence
  Synthetic construct
tgactgtaggcggggaagatga

SEQ ID NO: 25 - DNA, 20 bp, Artificial Sequence
  Synthetic construct
gagcaagctggaccttccat

SEQ ID NO: 26 - DNA, 16 bp, Artificial Sequence
  Synthetic construct
cctcaagcttgagngg

SEQ ID NO: 27 - DNA, 20 bp, Artificial Sequence
  Synthetic construct
tgcttgcaagcttgcaagca

SEQ ID NO: 28 - DNA, 18 bp, Artificial Sequence
  Synthetic construct
tgcttgcaagcttgcaag

SEQ ID NO: 29 - DNA, 16 bp, Artificial Sequence
  Synthetic construct
tgcttgcaagcttgca

SEQ ID NO: 30 - DNA, 19 bp, Artificial Sequence
  Synthetic construct
gcttgcaagcttgcaagca

SEQ ID NO: 31 - DNA, 18 bp, Artificial Sequence
  Synthetic construct
cttgcaagcttgcaagca

SEQ ID NO: 32 - DNA, 17 bp, Artificial Sequence
  Synthetic construct
ttgcaagcttgcaagca

SEQ ID NO: 33 - DNA, 20 bp, Artificial Sequence
  Synthetic construct
tgcttgcaagctagcaagca

SEQ ID NO: 34 - DNA, 20 bp, Artificial Sequence
  Synthetic construct
tgcttgcaagcttgctagca

SEQ ID NO: 35 - DNA, 20 bp, Artificial Sequence
  Synthetic construct
tgcttgacagcttgacagca

SEQ ID NO: 36 - DNA, 20 bp, Artificial Sequence
  Synthetic construct
tgcttagcagctatgcagca

SEQ ID NO: 37 - DNA, 20 bp, Artificial Sequence
  Synthetic construct
tgcaagcaagctagcaagca

SEQ ID NO: 38 - DNA, 25 bp, Artificial Sequence
  Synthetic construct
tgcaagcttgcaagcttgcaagctt

SEQ ID NO: 39 - DNA, 21 bp, Artificial Sequence
  Synthetic construct
tgctgcaagcttgcagatgat

SEQ ID NO: 40 - DNA, 19 bp, Artificial Sequence
  Synthetic construct
tgcttgcaagcttgcaagc

SEQ ID NO: 41 - DNA, 22 bp, Artificial Sequence
  Synthetic construct
tgcaagcttgcaagcttgcaat

SEQ ID NO: 42 - DNA, 14 bp, Artificial Sequence
  Synthetic construct
tgcttgcaagcttg

SEQ ID NO: 43 - DNA, 20 bp, Artificial Sequence
  Synthetic construct
agcttgcaagcttgcaagca

SEQ ID NO: 44 - DNA, 20 bp, Artificial Sequence
  Synthetic construct
tacttgcaagcttgcaagca

SEQ ID NO: 45 - DNA, 20 bp, Artificial Sequence
  Synthetic construct
tgattgcaagcttgcaagca

SEQ ID NO: 46 - DNA, 20 bp, Artificial Sequence
  Synthetic construct
aaattgcaagcttgcaagca

SEQ ID NO: 47 - DNA, 15 bp, Artificial Sequence
  Synthetic construct
tgctggaggggttgt

SEQ ID NO: 48 - DNA, 20 bp, Artificial Sequence
  Synthetic construct
aaattgacagcttgacagca

SEQ ID NO: 49 - DNA, 20 bp, Artificial Sequence
  Synthetic construct
tgattgacagcttgacagca

SEQ ID NO: 50 - DNA, 20 bp, Artificial Sequence
  Synthetic construct
tgattgacagattgacagca

SEQ ID NO: 51 - DNA, 20 bp, Artificial Sequence
  Synthetic construct
tgattgacagattgacagac

SEQ ID NO: 52 - DNA, 18 bp, Artificial Sequence
  Synthetic construct
tgctcctggaggggttgt

SEQ ID NO: 53 - DNA, 21 bp, Artificial Sequence
  Synthetic construct
tgcttgtcctggaggggttgt

SEQ ID NO: 54 - DNA, 24 bp, Artificial Sequence
  Synthetic construct
tgcttgacatcctggaggggttgt

SEQ ID NO: 55 - DNA, 33 bp, Artificial Sequence
  Synthetic construct
tgcttgacagcttgacagtcctggaggggttgt

SEQ ID NO: 56 - DNA, 30 bp, Artificial Sequence
  Synthetic construct
tgcttgacagcttgatcctggaggggttgt

SEQ ID NO: 57 - DNA, 27 bp, Artificial Sequence
  Synthetic construct
tgcttgacagcttcctggaggggttgt

SEQ ID NO: 58 - DNA, 30 bp, Artificial Sequence
  Synthetic construct
tgcttgacagcttgctcctggaggggttgt

SEQ ID NO: 59 - DNA, 33 bp, Artificial Sequence
  Synthetic construct
tgcttgacagcttgcttgtcctggaggggttgt

SEQ ID NO: 60 - DNA, 35 bp, Artificial Sequence
  Synthetic construct
tgcttgacagcttgacagcatcctggaggggttgt

SEQ ID NO: 61 - DNA, 35 bp, Artificial Sequence; identical to SEQ ID NO: 60
  Synthetic construct

SEQ ID NO: 62 - DNA, 32 bp, Artificial Sequence
  Synthetic construct
tgcttgacagcttgacagcatcctggaggggt

SEQ ID NO: 63 - DNA, 31 bp, Artificial Sequence
  Synthetic construct
tgcttgacagcttgacagcatcctggagggg

SEQ ID NO: 64 - DNA, 30 bp, Artificial Sequence
  Synthetic construct
tgcttgcaagcttgctcctggaggggttgt

SEQ ID NO: 65 - DNA, 27 bp, Artificial Sequence
  Synthetic construct
tgcttgcaagcttcctggaggggttgt

SEQ ID NO: 66 - DNA, 35 bp, Artificial Sequence
  Synthetic construct
tgcttgcaagcttgcaagcatcctggaggggttgt

SEQ ID NO: 67 - DNA, 35 bp, Artificial Sequence; identical to SEQ ID NO: 66
  Synthetic construct

SEQ ID NO: 68 - DNA, 35 bp, Artificial Sequence
  Synthetic construct
tgcttgcaagctagcaagcatcctggaggggttgt

SEQ ID NO: 69 - DNA, 35 bp, Artificial Sequence
  Synthetic construct
tgcttgcaagcttgctagcatcctggaggggttgt

SEQ ID NO: 70 - DNA, 35 bp, Artificial Sequence
  Synthetic construct
tgcttgcaagcttgctagcatcctggagnggttgt

SEQ ID NO: 71 - DNA, 35 bp, Artificial Sequence
  Synthetic construct
tcctggaggggttgttgcttgcaagcttgcaagca

SEQ ID NO: 72 - DNA, 22 bp, Artificial Sequence
  Synthetic construct
tgactgtgaacgttcgagatga

SEQ ID NO: 73 - DNA, 22 bp, Artificial Sequence
  Synthetic construct
tgactgtgaaccttagagatga

SEQ ID NO: 74 - DNA, 15 bp, Artificial Sequence
  Synthetic construct
tgctggaggggttgt

SEQ ID NO: 75 - DNA, 15 bp, Artificial Sequence
  Synthetic construct
tgctggaggggttgt

SEQ ID NO: 76 - DNA, 18 bp, Artificial Sequence
  Synthetic construct
tgcnnntggaggggttgt

SEQ ID NO: 77 - DNA, 17 bp, Artificial Sequence
  Synthetic construct
gctcctggaggggttgt

SEQ ID NO: 78 - DNA, 16 bp, Artificial Sequence
  Synthetic construct
ctcctggaggggttgt

SEQ ID NO: 79 - DNA, 18 bp, Artificial Sequence
  Synthetic construct
aaatcctggaggggttgt

SEQ ID NO: 80 - DNA, 15 bp, Artificial Sequence
  Synthetic construct
tcctggnggggttgt

SEQ ID NO: 81 - DNA, 16 bp, Artificial Sequence
  Synthetic construct
tcctggnnggggttgt

SEQ ID NO: 82 - DNA, 18 bp, Artificial Sequence
  Synthetic construct
tgctcctggaggggttgt

SEQ ID NO: 83 - DNA, 53 bp, Artificial Sequence
  Synthetic construct
tgcnnnnnnnnnnnnnnnnnnnnnnnnnnnnnnnnnnnnnnnnnnnnnnnnnn

SEQ ID NO: 84 - DNA, 54 bp, Artificial Sequence
  Synthetic construct
tgcnnnnnnnnnnnnnnnnnnnnnnnnnnnnnnnnnnnnnnnnnnnnnnnnnna

SEQ ID NO: 85 - DNA, 55 bp, Artificial Sequence
  Synthetic construct
tgcnnnnnnnnnnnnnnnnnnnnnnnnnnnnnnnnnnnnnnnnnnnnnnnnnnca

SEQ ID NO: 86 - DNA, 56 bp, Artificial Sequence
  Synthetic construct
tgcnnnnnnnnnnnnnnnnnnnnnnnnnnnnnnnnnnnnnnnnnnnnnnnnnngca

SEQ ID NO: 87 - DNA, 16 bp, Artificial Sequence
  Synthetic construct
tcctggaggggttgtt

SEQ ID NO: 88 - DNA, 16 bp, Artificial Sequence
  Synthetic construct
tcctggaggggttgtt

SEQ ID NO: 89 - DNA, 16 bp, Artificial Sequence
  Synthetic construct
tcctggaggggttgtt

SEQ ID NO: 90 - DNA, 16 bp, Artificial Sequence
  Synthetic construct
tcctggaggggttgtt